(19) United States
(12) Patent Application Publication
(10) Pub. No.: US 2026/0060800 A1
(43) Pub. Date: Mar. 5, 2026

(54) ACCOMMODATING INTRAOCULAR LENSES SUPPORTING MYOPIC SHIFT IN REFRACTION DURING ACCOMMODATIVE REFLEX

(71) Applicant: OnPoint Vision, Inc., Aliso Viejo, CA (US)

(72) Inventor: Kevin J. Cady, Franklin, TN (US)

(21) Appl. No.: 19/293,731

(22) Filed: Aug. 7, 2025

Related U.S. Application Data
(60) Provisional application No. 63/687,993, filed on Aug. 28, 2024.

(51) Int. Cl. A61F 2/16

(52) U.S. Cl. CPC A61F 2/1648; A61F 2210/0057

(57) ABSTRACT

An accommodating intraocular lens system includes a posterior intraocular lens having a first optical lens and first haptics. The accommodating intraocular lens system also includes an anterior intraocular lens having a second optical lens and second haptics, where the second haptics are attached to the posterior intraocular lens. The posterior intraocular lens is configured to remain in a substantially fixed position within an eye in an absence of accommodative reflex. The anterior intraocular lens is configured to at least one of change shape or change position during accommodative reflex in order to increase a dioptric power of the accommodating intraocular lens system. In some cases, the posterior intraocular lens May be more rigid than the anterior intraocular lens, and the anterior intraocular lens may be more flexible or elastic than the posterior intraocular lens.

### CROSS-REFERENCE TO RELATED APPLICATION AND PRIORITY CLAIM

This application claims priority under 35 U.S.C. § 119(e) to U.S. Provisional Patent Application No. 63/687,993 filed on Aug. 28, 2024, which is hereby incorporated by reference in its entirety.

### TECHNICAL FIELD

This disclosure relates generally to implantable optical devices. More specifically, this disclosure relates to accommodating intraocular lenses supporting myopic shift in refraction during accommodative reflex.

### BACKGROUND

There are various conditions that can affect a person's vision, and a number of these conditions can affect the lenses in a person's eyes. For example, people can suffer from cataracts and presbyopia, separately or in combination. With respect to cataracts, the crystalline lens of a person's eye can lose clarity and become more opaque. This can cause the person's vision to become cloudy or to otherwise decrease the person's vision. With respect to presbyopia, a healthy human eye can focus on objects at both far and near distances, and the ability of the eye to change back and forth from near vision to far vision is called accommodation. Presbyopia refers to the loss of accommodation and results in the inability to focus on nearby objects, which is why people often need reading glasses as they age.

### SUMMARY

This disclosure relates to accommodating intraocular lenses supporting myopic shift in refraction during accommodative reflex.

In a first embodiment, an accommodating intraocular lens system includes a posterior intraocular lens having a first optical lens and first haptics. The accommodating intraocular lens system also includes an anterior intraocular lens having a second optical lens and second haptics, where the second haptics are attached to the posterior intraocular lens. The posterior intraocular lens is configured to remain in a substantially fixed position within an eye in an absence of accommodative reflex. The anterior intraocular lens is configured to at least one of change shape or change position during accommodative reflex in order to increase a dioptric power of the accommodating intraocular lens system.

Any single one or any combination of the following features may be used with the first embodiment. The posterior intraocular lens may be more rigid than the anterior intraocular lens, and the anterior intraocular lens may be more flexible or elastic than the posterior intraocular lens. A portion of the anterior intraocular lens may be configured to form a larger gap between the anterior intraocular lens and the posterior intraocular lens during accommodative reflex, and the portion of the anterior intraocular lens may be configured to form a smaller gap or no gap between the anterior intraocular lens and the posterior intraocular lens otherwise. The anterior intraocular lens may be configured to bulge outward during accommodative reflex to form the larger gap. The posterior intraocular lens may include acrylic, and the anterior intraocular lens may include silicone. The second haptics may include projections extending from the second optical lens and ridges configured to contact at least one edge of the posterior intraocular lens. The first optical lens may represent a plano optical lens. The first and second optical lenses may collectively provide a desired amount of dioptric power of the accommodating intraocular lens system. The change(s) in the shape and/or the position of the anterior intraocular lens may be defined by (i) an increase in separation of the second optical lens from the first optical lens and (ii) an increase in bending of the second haptics backwards during accommodative reflex.

In a second embodiment, an accommodating intraocular lens system includes a posterior intraocular lens having a first optical lens and first haptics. The accommodating intraocular lens system also includes an anterior intraocular lens having a second optical lens and second haptics, where the second haptics are attached to the posterior intraocular lens. The posterior intraocular lens is configured to be moved forward and backward in an eye based on a presence or absence of accommodative reflex. The posterior intraocular lens is more rigid than the anterior intraocular lens, and the anterior intraocular lens is more flexible or elastic than the posterior intraocular lens. The anterior intraocular lens is configured to at least one of change shape or change position during accommodative reflex in order to increase a dioptric power of the accommodating intraocular lens system.

Any single one or any combination of the following features may be used with the second embodiment. A portion of the anterior intraocular lens may be configured to form a larger gap between the anterior intraocular lens and the posterior intraocular lens during accommodative reflex, and the portion of the anterior intraocular lens may be configured to form a smaller gap or no gap between the anterior intraocular lens and the posterior intraocular lens otherwise. The anterior intraocular lens may be configured to bulge outward to form the larger gap. The posterior intraocular lens may include acrylic, and the anterior intraocular lens may include silicone. The second haptics may include projections extending from the second optical lens and ridges configured to contact at least one edge of the posterior intraocular lens.

The first optical lens may represent a plano optical lens. The first and second optical lenses may collectively provide a desired amount of dioptric power of the accommodating intraocular lens system. The posterior intraocular lens may be configured to remain in a substantially fixed position within an eye in an absence of accommodative reflex.

In a third embodiment, an accommodating intraocular lens system includes a posterior intraocular lens having a first optical lens and first haptics. The accommodating intraocular lens system also includes an anterior intraocular lens having a second optical lens and second haptics, where the second haptics are attached to the posterior intraocular lens. The posterior intraocular lens is configured to be moved forward and backward in an eye based on a presence or absence of accommodative reflex. The posterior intraocular lens is more rigid than the anterior intraocular lens, and the anterior intraocular lens is more flexible or elastic than the posterior intraocular lens. A portion of the anterior intraocular lens is configured to form a larger gap between the anterior intraocular lens and the posterior intraocular lens during accommodative reflex, and the portion of the anterior intraocular lens is configured to form a smaller gap or no gap between the anterior intraocular lens and the posterior intraocular lens otherwise. The anterior intraocular lens is configured to move or bulge outward to form the larger gap.

Any single one or any combination of the following features may be used with the third embodiment. The posterior intraocular lens may include acrylic, and the anterior intraocular lens may include silicone. The second haptics may include projections extending from the second optical lens and ridges configured to contact at least one edge of the posterior intraocular lens.

Other technical features may be readily apparent to one skilled in the art from the following figures, descriptions, and claims.

### BRIEF DESCRIPTION OF THE DRAWINGS

For a more complete understanding of this disclosure and its features, reference is now made to the following description, taken in conjunction with the accompanying drawings, in which:

FIGS. 1A through 1D illustrate a first example accommodating intraocular lens having a dual-lens system supporting myopic shift in refraction during accommodative reflex according to this disclosure;

FIGS. 2A through 2D illustrate a second example accommodating intraocular lens having a dual-lens system supporting myopic shift in refraction during accommodative reflex according to this disclosure;

FIGS. 3A through 3D illustrate a third example accommodating intraocular lens having a dual-lens system supporting myopic shift in refraction during accommodative reflex according to this disclosure;

FIGS. 4A through 4D illustrate a fourth example accommodating intraocular lens having a dual-lens system supporting myopic shift in refraction during accommodative reflex according to this disclosure;

FIGS. 5A through 5D illustrate a fifth example accommodating intraocular lens having a dual-lens system supporting myopic shift in refraction during accommodative reflex according to this disclosure;

FIGS. 6A and 6B illustrate a first example of an anterior intraocular lens that may be used in a dual-lens system supporting myopic shift in refraction during accommodative reflex according to this disclosure;

FIGS. 7A through 7C illustrate a second example of an anterior intraocular lens that may be used in a dual-lens system supporting myopic shift in refraction during accommodative reflex according to this disclosure;

FIGS. 8A through 8C illustrate a third example of an anterior intraocular lens that may be used in a dual-lens system supporting myopic shift in refraction during accommodative reflex according to this disclosure;

FIGS. 9A through 9C illustrate a fourth example of an anterior intraocular lens that may be used in a dual-lens system supporting myopic shift in refraction during accommodative reflex according to this disclosure;

FIGS. 10A through 10C illustrate a fifth example of an anterior intraocular lens that may be used in a dual-lens system supporting myopic shift in refraction during accommodative reflex according to this disclosure;

FIGS. 11A through 11C illustrate a sixth example of an anterior intraocular lens that may be used in a dual-lens system supporting myopic shift in refraction during accommodative reflex according to this disclosure;

FIGS. 12A through 12C illustrate a seventh example of an anterior intraocular lens that may be used in a dual-lens system supporting myopic shift in refraction during accommodative reflex according to this disclosure;

FIGS. 13A through 13C illustrate an eighth example of an anterior intraocular lens that may be used in a dual-lens system supporting myopic shift in refraction during accommodative reflex according to this disclosure;

FIGS. 14A through 14C illustrate a ninth example of an anterior intraocular lens that may be used in a dual-lens system supporting myopic shift in refraction during accommodative reflex according to this disclosure;

FIGS. 15A through 15C illustrate a tenth example of an anterior intraocular lens that may be used in a dual-lens system supporting myopic shift in refraction during accommodative reflex according to this disclosure;

FIGS. 16A through 16C illustrate an eleventh example of an anterior intraocular lens that may be used in a dual-lens system supporting myopic shift in refraction during accommodative reflex according to this disclosure; and

FIGS. 17A and 17B illustrate example operation of an accommodating intraocular lens having a dual-lens system supporting myopic shift in refraction during accommodative reflex according to this disclosure.

### DETAILED DESCRIPTION

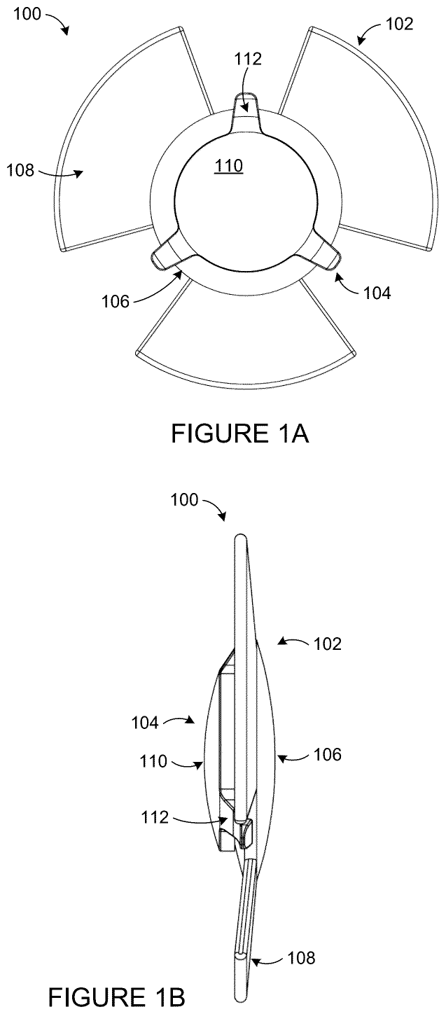

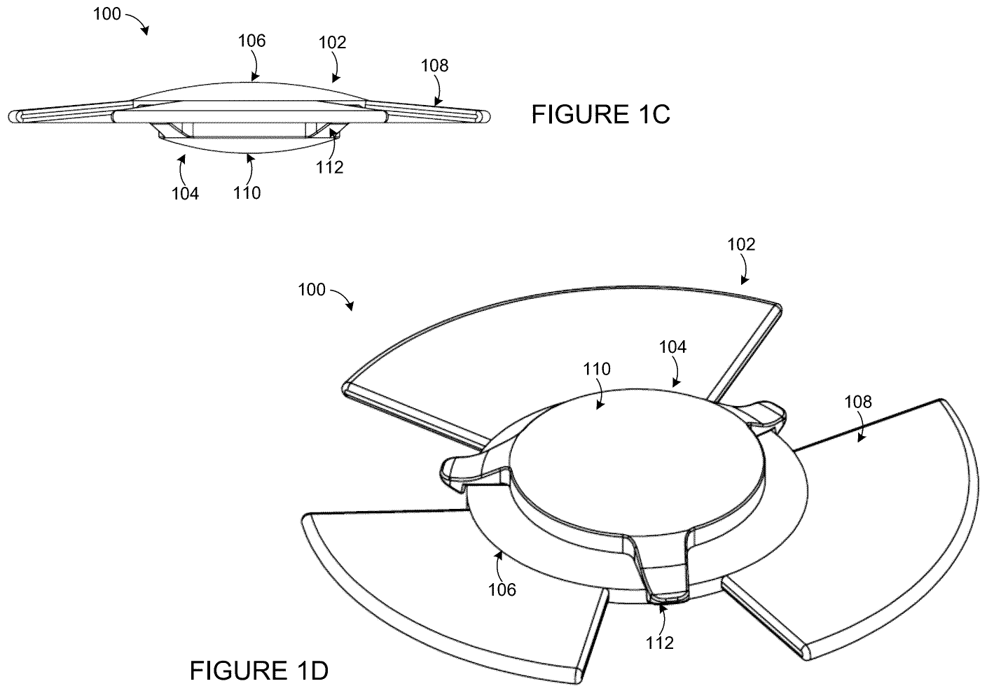

FIGS. 1A through 17B, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

As noted above, there are various conditions that can affect a person's vision, and a number of these conditions can affect the lenses in a person's eyes. For example, people can suffer from cataracts and presbyopia, separately or in combination. With respect to cataracts, the crystalline lens of a person's eye can lose clarity and become more opaque. This can cause the person's vision to become cloudy or to otherwise decrease the person's vision. With respect to presbyopia, a healthy human eye can focus on objects at both far and near distances, and the ability of the eye to change back and forth from near vision to far vision is called accommodation. Presbyopia refers to the loss of accommodation and results in the inability to focus on nearby objects, which is why people often need reading glasses as they age.

During a conventional cataract surgery, the crystalline lens in a patient's eye is removed and replaced with an artificial intraocular lens (IOL). For example, the crystalline lens core is often removed by phacoemulsification through a curvilinear capsularhexis, leaving intact the thin walls of the anterior and posterior capsules together with zonular ligament connections to the ciliary body and ciliary muscles. In most cases, this allows an artificial intraocular lens to be placed within the remaining portion of the capsular bag of the patient's eye. Depending on the design, some artificial intraocular lenses are “single-vision” and only restore the patient's ability to focus at a fixed distance. Other artificial intraocular lenses are “accommodative” or “multi-vision” and provide some ability for the patient to focus at both closer and farther distances. Often times, however, the patient's ability to focus at closer distances is limited, and the patient with uncorrected near vision (UCNV) may achieve a limited near vision acuity range.

This disclosure provides various accommodating intraocular lenses supporting myopic shift in refraction during accommodative reflex. As described in more detail below, each accommodating intraocular lens represents an integrated, dual-optic lens system design that includes a primary posterior intraocular lens and a secondary anterior intraocular lens. The anterior intraocular lens is positioned anteriorly or in front of the posterior intraocular lens. In some cases, the posterior intraocular lens may represent a biconvex base intraocular lens, and the anterior intraocular lens may represent a convex/concave single-vision intraocular lens. Also, in some cases, the anterior intraocular lens can be fused with or otherwise permanently attached to the posterior intraocular lens. In addition, in some cases, the posterior intraocular lens can be made of a more rigid material like acrylic or stiffer silicone, and the anterior intraocular lens can be made of a more flexible material like more flexible silicone. During use, the accommodating intraocular lens may typically be designed to provide focus at a farther distance. When the patient attempts to focus at a closer distance, a portion of the anterior intraocular lens can separate from the posterior intraocular lens, thereby providing a greater increase in optical or dioptric power and a greater increase in depth of focus than are achievable through only movement of the posterior intraocular lens.

In this way, the accommodating intraocular lenses described below can be used to provide accommodation in a patient's eye, allowing the patient's eye to focus at closer and farther distances. Also, the separation of the anterior and posterior intraocular lenses during accommodation can actually increase the optical or dioptric power of the anterior and posterior intraocular lenses and provide improved vision during accommodation, even in excess of the optical or dioptric power expected from the combination of the anterior and posterior intraocular lenses. In some instances, patients may achieve improved near vision as a result of this separation of the anterior and posterior intraocular lenses.

FIGS. 1A through 1D illustrate a first example accommodating intraocular lens 100 having a dual-lens system supporting myopic shift in refraction during accommodative reflex according to this disclosure. As can be seen in FIGS. 1A through 1D, the accommodating intraocular lens 100 includes a posterior intraocular lens 102 and an anterior intraocular lens 104. The posterior intraocular lens 102 includes an optical lens 106 and one or more haptics 108. The optical lens 106 of the posterior intraocular lens 102 receives light entering a patient's eye and adjusts the light, such as by providing a refractive correction or other refractive or vision adjustment in the patient's eye. The haptics 108 of the posterior intraocular lens 102 help to hold the posterior intraocular lens 102 within a capsular bag of the patient's eye or to otherwise hold the posterior intraocular lens 102 in place within the patient's eye. This helps to position the optical lens 106 of the posterior intraocular lens 102 in a desired location within the eye.

The posterior intraocular lens 102 can be formed from any suitable material(s), such as acrylic, stiffer silicone, or other more rigid material(s). The posterior intraocular lens 102 can also be formed in any suitable manner, such as by using a mold or lathe cut manufacturing process. Different posterior intraocular lenses 102 can be designed and manufactured to provide a wide range of diopters, and each posterior intraocular lens 102 can be designed to correct any suitable refractive error(s) or provide other suitable vision correction or adjustment (such as high magnification or multifocality). In other cases, however, the posterior intraocular lens 102 may be neutral, meaning the optical lens 106 may represent a plano optical lens that provides no spherical power. Example types of common refractive errors that can be corrected include myopia, hyperopia, and astigmatism. In some cases, the haptics 108 may be configured to move the optical lens 106 forward or backward within the patient's eye. Even small changes in the forward or backward positional movement of the posterior intraocular lens 102 can create a myopic shift in refraction within the patient's eye.

The anterior intraocular lens 104 includes an optical lens 110 and multiple haptics 112. The optical lens 110 of the anterior intraocular lens 104 receives light entering the eye and may adjust the light prior to reaching the posterior intraocular lens 102, such as by providing another refractive correction or other refractive or vision adjustment in the patient's eye. The haptics 112 of the anterior intraocular lens 104 help to attach the anterior intraocular lens 104 to the posterior intraocular lens 102. In this example, the haptics 112 are formed by projections that extend from the sides of the optical lens 110. The projection of each haptic 112 includes a ridge, and multiple ridges of multiple haptics 112 can be used to contact or capture one or more edges of the underlying posterior intraocular lens 102. This can help to center the anterior intraocular lens 104 on the posterior intraocular lens 102. The haptics 112 of the anterior intraocular lens 104 can be fused or otherwise attached to the posterior intraocular lens 102 to help hold the anterior intraocular lens 104 in place.

The anterior intraocular lens 104 can be formed from any suitable material(s), such as more flexible silicone or other more flexible or clastic material(s). The anterior intraocular lens 104 can also be formed in any suitable manner, such as by using a mold or lathe cut manufacturing process. Different anterior intraocular lenses 104 can be designed and manufactured to provide a wide range of diopters, and each anterior intraocular lens 104 can be designed to correct any suitable refractive error(s) or provide other suitable vision correction or adjustment (such as high magnification or multifocality).

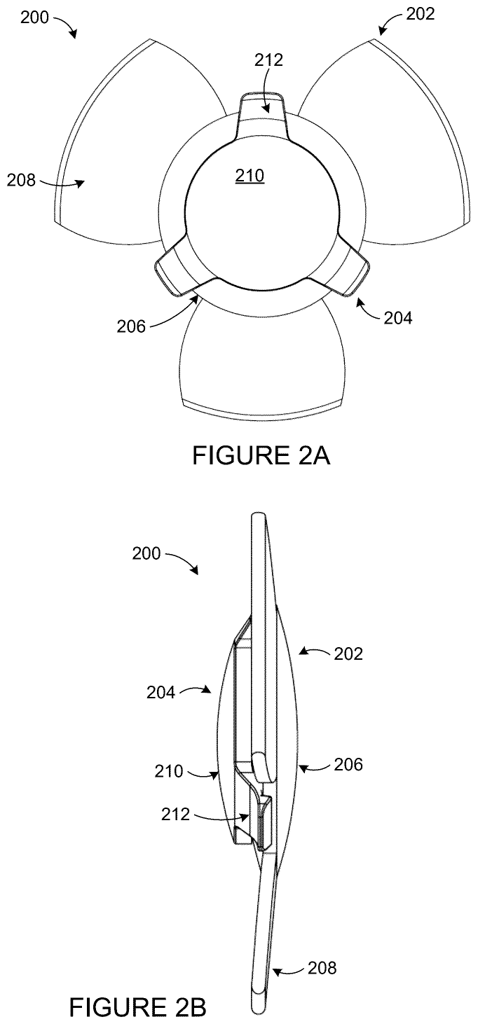

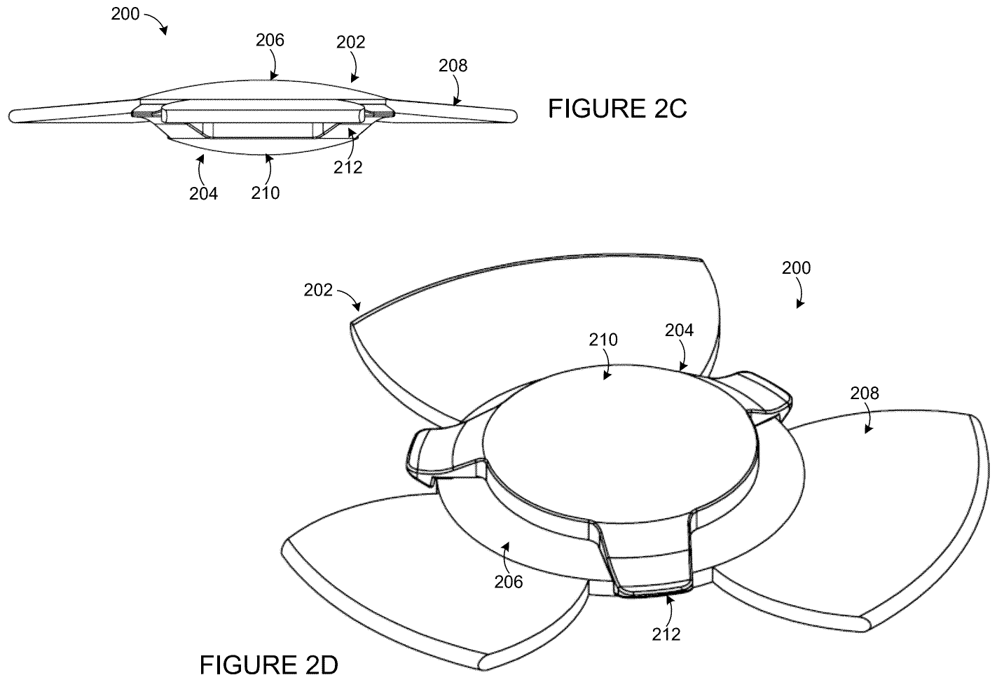

FIGS. 2A through 2D illustrate a second example accommodating intraocular lens 200 having a dual-lens system supporting myopic shift in refraction during accommodative reflex according to this disclosure. As can be seen in FIGS. 2A through 2D, the accommodating intraocular lens 200 includes a posterior intraocular lens 202 and an anterior intraocular lens 204. The posterior intraocular lens 202 includes an optical lens 206 and one or more haptics 208. The optical lens 206 of the posterior intraocular lens 202 receives light entering a patient's eye and adjusts the light, such as by providing a refractive correction or other refractive or vision adjustment in the patient's eye. The haptics 208 of the posterior intraocular lens 202 help to hold the posterior intraocular lens 202 within a capsular bag of the patient's eye or to otherwise hold the posterior intraocular lens 202 in place within the patient's eye. This helps to position the optical lens 206 of the posterior intraocular lens 202 in a desired location within the eye.

The posterior intraocular lens 202 can be formed from any suitable material(s), such as acrylic, stiffer silicone, or other more rigid material(s). The posterior intraocular lens 202 can also be formed in any suitable manner, such as by using a mold or lathe cut manufacturing process. Different posterior intraocular lenses 202 can be designed and manufactured to provide a wide range of diopters, and each posterior intraocular lens 202 can be designed to correct any suitable refractive error(s) or provide other suitable vision correction or adjustment (such as high magnification or multifocality). In other cases, however, the posterior intraocular lens 202 may be neutral, meaning the optical lens 206 may represent a plano optical lens that provides no spherical power. In some cases, the haptics 208 may be configured to move the optical lens 206 forward or backward within the patient's eye. Even small changes in the forward or backward positional movement of the posterior intraocular lens 202 can create a myopic shift in refraction within the patient's eye.

The anterior intraocular lens 204 includes an optical lens 210 and multiple haptics 212. The optical lens 210 of the anterior intraocular lens 204 receives light entering the eye and may adjust the light prior to reaching the posterior intraocular lens 202, such as by providing another refractive correction or other refractive or vision adjustment in the patient's eye. The haptics 212 of the anterior intraocular lens 204 help to attach the anterior intraocular lens 204 to the posterior intraocular lens 202. In this example, the haptics 212 are formed by projections that extend from the sides of the optical lens 210. The projection of each haptic 212 includes a ridge, and multiple ridges of multiple haptics 212 can be used to contact or capture one or more edges of the underlying posterior intraocular lens 202. This can help to center the anterior intraocular lens 204 on the posterior intraocular lens 202. The haptics 212 of the anterior intraocular lens 204 can be fused or otherwise attached to the posterior intraocular lens 202 to help hold the anterior intraocular lens 204 in place.

The anterior intraocular lens 204 can be formed from any suitable material(s), such as more flexible silicone or other more flexible or elastic material(s). The anterior intraocular lens 204 can also be formed in any suitable manner, such as by using a mold or lathe cut manufacturing process. Different anterior intraocular lenses 204 can be designed and manufactured to provide a wide range of diopters, and each anterior intraocular lens 204 can be designed to correct any suitable refractive error(s) or provide other suitable vision correction or adjustment (such as high magnification or multifocality).

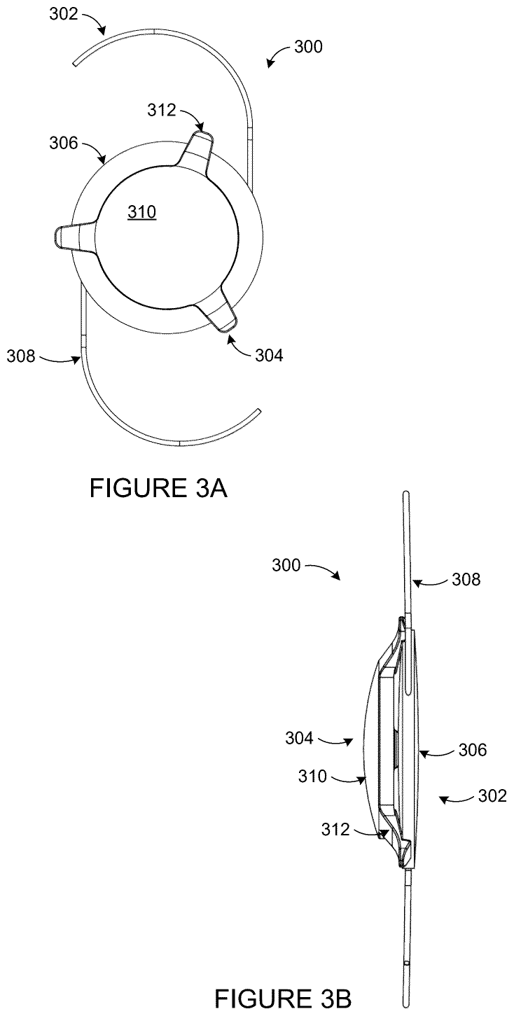

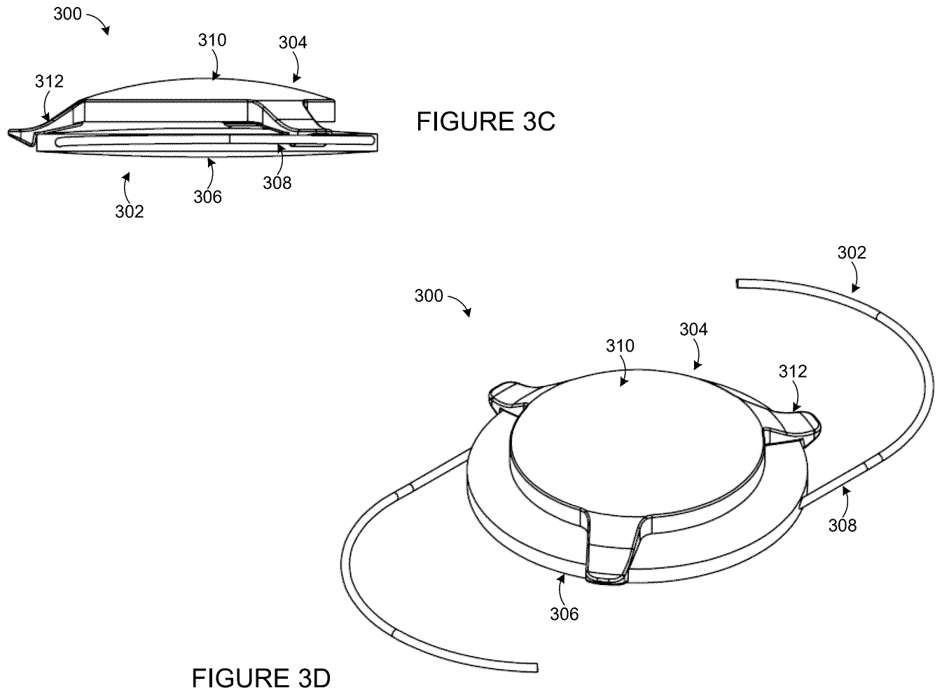

FIGS. 3A through 3D illustrate a third example accommodating intraocular lens 300 having a dual-lens system supporting myopic shift in refraction during accommodative reflex according to this disclosure. As can be seen in FIGS. 3A through 3D, the accommodating intraocular lens 300 includes a posterior intraocular lens 302 and an anterior intraocular lens 304. The posterior intraocular lens 302 includes an optical lens 306 and one or more haptics 308. The optical lens 306 of the posterior intraocular lens 302 receives light entering a patient's eye and adjusts the light, such as by providing a refractive correction or other refractive or vision adjustment in the patient's eye. The haptics 308 of the posterior intraocular lens 302 help to hold the posterior intraocular lens 302 within a capsular bag of the patient's eye or to otherwise hold the posterior intraocular lens 302 in place within the patient's eye. This helps to position the optical lens 306 of the posterior intraocular lens 302 in a desired location within the eye.

The posterior intraocular lens 302 can be formed from any suitable material(s), such as acrylic, stiffer silicone, or other more rigid material(s). The posterior intraocular lens 302 can also be formed in any suitable manner, such as by using a mold or lathe cut manufacturing process. Different posterior intraocular lenses 302 can be designed and manufactured to provide a wide range of diopters, and each posterior intraocular lens 302 can be designed to correct any suitable refractive error(s) or provide other suitable vision correction or adjustment (such as high magnification or multifocality). In other cases, however, the posterior intraocular lens 302 may be neutral, meaning the optical lens 306 may represent a plano optical lens that provides no spherical power. In some cases, the haptics 308 may be configured to move the optical lens 306 forward or backward within the patient's eye. Even small changes in the forward or backward positional movement of the posterior intraocular lens 302 can create a myopic shift in refraction within the patient's eye.

The anterior intraocular lens 304 includes an optical lens 310 and multiple haptics 312. The optical lens 310 of the anterior intraocular lens 304 receives light entering the eye and may adjust the light prior to reaching the posterior intraocular lens 302, such as by providing another refractive correction or other refractive or vision adjustment in the patient's eye. The haptics 312 of the anterior intraocular lens 304 help to attach the anterior intraocular lens 304 to the posterior intraocular lens 302. In this example, the haptics 312 are formed by projections that extend from the sides of the optical lens 310. The projection of each haptic 312 includes a ridge, and multiple ridges of multiple haptics 312 can be used to contact or capture one or more edges of the underlying posterior intraocular lens 302. This can help to center the anterior intraocular lens 304 on the posterior intraocular lens 302. The haptics 312 of the anterior intraocular lens 304 can be fused or otherwise attached to the posterior intraocular lens 302 to help hold the anterior intraocular lens 304 in place.

The anterior intraocular lens 304 can be formed from any suitable material(s), such as more flexible silicone or other more flexible or clastic material(s). The anterior intraocular lens 304 can also be formed in any suitable manner, such as by using a mold or lathe cut manufacturing process. Different anterior intraocular lenses 304 can be designed and manufactured to provide a wide range of diopters, and each anterior intraocular lens 304 can be designed to correct any suitable refractive error(s) or provide other suitable vision correction or adjustment (such as high magnification or multifocality).

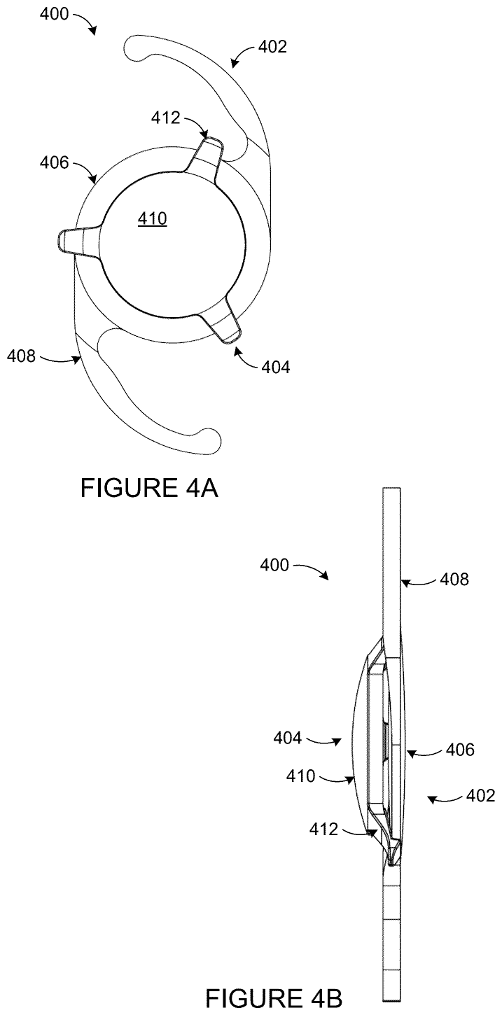

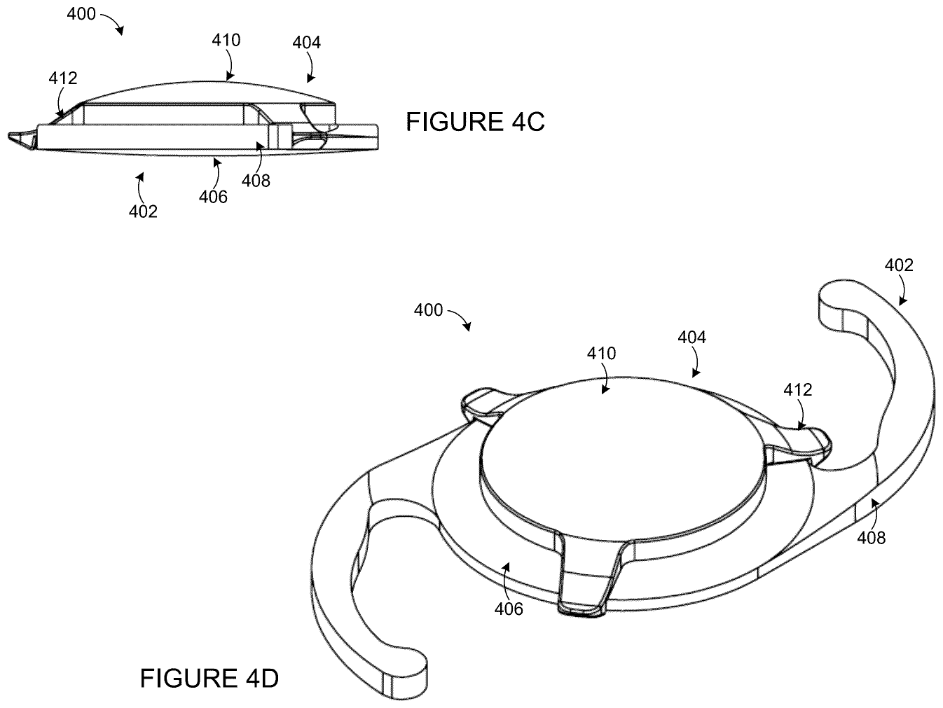

FIGS. 4A through 4D illustrate a fourth example accommodating intraocular lens 400 having a dual-lens system supporting myopic shift in refraction during accommodative reflex according to this disclosure. As can be seen in FIGS. 4A through 4D, the accommodating intraocular lens 400 includes a posterior intraocular lens 402 and an anterior intraocular lens 404. The posterior intraocular lens 402 includes an optical lens 406 and one or more haptics 408. The optical lens 406 of the posterior intraocular lens 402 receives light entering a patient's eye and adjusts the light, such as by providing a refractive correction or other refractive or vision adjustment in the patient's eye. The haptics 408 of the posterior intraocular lens 402 help to hold the posterior intraocular lens 402 within a capsular bag of the patient's eye or to otherwise hold the posterior intraocular lens 402 in place within the patient's eye. This helps to position the optical lens 406 of the posterior intraocular lens 402 in a desired location within the eye.

The posterior intraocular lens 402 can be formed from any suitable material(s), such as acrylic, stiffer silicone, or other more rigid material(s). The posterior intraocular lens 402 can also be formed in any suitable manner, such as by using a mold or lathe cut manufacturing process. Different posterior intraocular lenses 402 can be designed and manufactured to provide a wide range of diopters, and each posterior intraocular lens 402 can be designed to correct any suitable refractive error(s) or provide other suitable vision correction or adjustment (such as high magnification or multifocality). In other cases, however, the posterior intraocular lens 402 may be neutral, meaning the optical lens 406 may represent a plano optical lens that provides no spherical power. In some cases, the haptics 408 may be configured to move the optical lens 406 forward or backward within the patient's eye. Even small changes in the forward or backward positional movement of the posterior intraocular lens 402 can create a myopic shift in refraction within the patient's eye.

The anterior intraocular lens 404 includes an optical lens 410 and multiple haptics 412. The optical lens 410 of the anterior intraocular lens 404 receives light entering the eye and may adjust the light prior to reaching the posterior intraocular lens 402, such as by providing another refractive correction or other refractive or vision adjustment in the patient's eye. The haptics 412 of the anterior intraocular lens 404 help to attach the anterior intraocular lens 404 to the posterior intraocular lens 402. In this example, the haptics 412 are formed by projections that extend from the sides of the optical lens 410. The projection of each haptic 412 includes a ridge, and multiple ridges of multiple haptics 412 can be used to contact or capture one or more edges of the underlying posterior intraocular lens 402. This can help to center the anterior intraocular lens 404 on the posterior intraocular lens 402. The haptics 412 of the anterior intraocular lens 404 can be fused or otherwise attached to the posterior intraocular lens 402 to help hold the anterior intraocular lens 404 in place.

The anterior intraocular lens 404 can be formed from any suitable material(s), such as more flexible silicone or other more flexible or clastic material(s). The anterior intraocular lens 404 can also be formed in any suitable manner, such as by using a mold or lathe cut manufacturing process. Different anterior intraocular lenses 404 can be designed and manufactured to provide a wide range of diopters, and each anterior intraocular lens 404 can be designed to correct any suitable refractive error(s) or provide other suitable vision correction or adjustment (such as high magnification or multifocality).

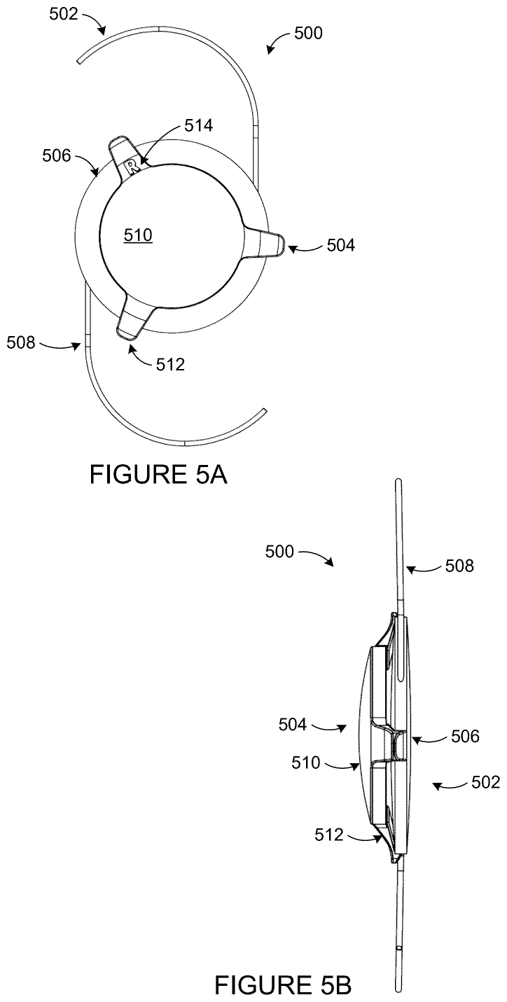

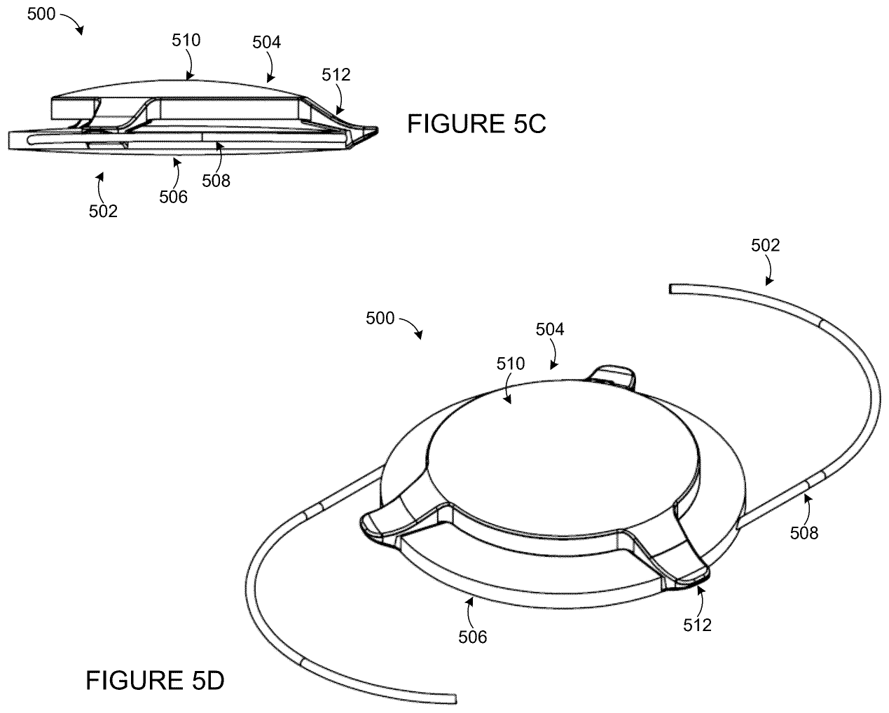

FIGS. 5A through 5D illustrate a fifth example accommodating intraocular lens 500 having a dual-lens system supporting myopic shift in refraction during accommodative reflex according to this disclosure. As can be seen in FIGS. 5A through 5D, the accommodating intraocular lens 500 includes a posterior intraocular lens 502 and an anterior intraocular lens 504. The posterior intraocular lens 502 includes an optical lens 506 and one or more haptics 508. The optical lens 506 of the posterior intraocular lens 502 receives light entering a patient's eye and adjusts the light, such as by providing a refractive correction or other refractive or vision adjustment in the patient's eye. The haptics 508 of the posterior intraocular lens 502 help to hold the posterior intraocular lens 502 within a capsular bag of the patient's eye or to otherwise hold the posterior intraocular lens 502 in place within the patient's eye. This helps to position the optical lens 506 of the posterior intraocular lens 502 in a desired location within the eye.

The posterior intraocular lens 502 can be formed from any suitable material(s), such as acrylic, stiffer silicone, or other more rigid material(s). The posterior intraocular lens 502 can also be formed in any suitable manner, such as by using a mold or lathe cut manufacturing process. Different posterior intraocular lenses 502 can be designed and manufactured to provide a wide range of diopters, and each posterior intraocular lens 502 can be designed to correct any suitable refractive error(s) or provide other suitable vision correction or adjustment (such as high magnification or multifocality). In other cases, however, the posterior intraocular lens 502 may be neutral, meaning the optical lens 506 may represent a plano optical lens that provides no spherical power. In some cases, the haptics 508 may be configured to move the optical lens 506 forward or backward within the patient's eye. Even small changes in the forward or backward positional movement of the posterior intraocular lens 502 can create a myopic shift in refraction within the patient's eye.

The anterior intraocular lens 504 includes an optical lens 510 and multiple haptics 512. The optical lens 510 of the anterior intraocular lens 504 receives light entering the eye and may adjust the light prior to reaching the posterior intraocular lens 502, such as by providing another refractive correction or other refractive or vision adjustment in the patient's eye. The haptics 512 of the anterior intraocular lens 504 help to attach the anterior intraocular lens 504 to the posterior intraocular lens 502. In this example, the haptics 512 are formed by projections that extend from the sides of the optical lens 510. The projection of each haptic 512 includes a ridge, and multiple ridges of multiple haptics 512 can be used to contact or capture one or more edges of the underlying posterior intraocular lens 502. This can help to center the anterior intraocular lens 504 on the posterior intraocular lens 502. The haptics 512 of the anterior intraocular lens 504 can be fused or otherwise attached to the posterior intraocular lens 502 to help hold the anterior intraocular lens 504 in place.

The anterior intraocular lens 504 can be formed from any suitable material(s), such as more flexible silicone or other more flexible or elastic material(s). The anterior intraocular lens 504 can also be formed in any suitable manner, such as by using a mold or lathe cut manufacturing process. Different anterior intraocular lenses 504 can be designed and manufactured to provide a wide range of diopters, and each anterior intraocular lens 504 can be designed to correct any suitable refractive error(s) or provide other suitable vision correction or adjustment (such as high magnification or multifocality).

In this example, at least one alignment marking 514 is provided, such as on one or more of the haptics 512. In this particular example, the alignment marking 514 represents the letter “R,” although any other suitable marking(s) may be used here in any suitable location(s). The alignment marking(s) 514 can be used to identify the proper orientation of the accommodating intraocular lens 500, such as by identifying the haptic 512 that is to be positioned on the right side of the accommodating intraocular lens 500 (from the perspective of the surgeon or other medical personnel) after implantation. Although not shown in the other figures, any of the accommodating intraocular lenses 100, 200, 300, 400 described above may include at least one alignment marking 514.

In each of these accommodating intraocular lenses 100, 200, 300, 400, 500, the anterior intraocular lens 104, 204, 304, 404, 504 can have a convex anterior surface and a concave posterior surface. Also, in each of these accommodating intraocular lenses 100, 200, 300, 400, 500, the posterior intraocular lens 102, 202, 302, 402, 502 can have a convex anterior surface and a convex posterior surface, thereby forming a biconvex lens. The pair of intraocular lenses 102-104, 202-204, 302-304, 402-404, 502-504 can be presented as a single intraocular lens system.

Each anterior intraocular lens 104, 204, 304, 404, 504 may be formed from a different material or materials than the corresponding posterior intraocular lens 102, 202, 302, 402, 502. For example, each posterior intraocular lens 102, 202, 302, 402, 502 may be formed from acrylic, stiffer silicone, or other more rigid material(s) and therefore provide a more rigid platform. Each anterior intraocular lens 104, 204, 304, 404, 504 may be formed from more flexible silicone or other more flexible or elastic material(s). As a result, the lenses in each pair of lenses 102-104, 202-204, 302-304, 402-404, 502-504 possess different elastic properties, where the anterior intraocular lens 104, 204, 304, 404, 504 can have a more elastic property than the associated posterior intraocular lens 102, 202, 302, 402, 502. This allows for an increase in anterior movement of the anterior intraocular lens 104, 204, 304, 404, 504 during accommodative reflex, while the more rigid posterior intraocular lens 102, 202, 302, 402, 502 remains less flexible.

The difference in elastic properties of the lenses allows for an increase in separational spacing of the anterior lenticular surface (front surface) of the posterior intraocular lens 102, 202, 302, 402, 502 to the posterior lenticular surface (back surface) of the more flexible anterior intraocular lens 104, 204, 304, 404, 504. This means that a gap can form between a central portion of the posterior intraocular lens 102, 202, 302, 402, 502 and a central portion of the anterior intraocular lens 104, 204, 304, 404, 504 during accommodative reflex. For example, a portion of the anterior intraocular lens 104, 204, 304, 404, 504 can form a larger gap between the anterior intraocular lens 104, 204, 304, 404, 504 and the posterior intraocular lens 102, 202, 302, 402, 502 during accommodative reflex. The portion of the anterior intraocular lens 104, 204, 304, 404, 504 can form a smaller gap or no gap between the anterior intraocular lens 104, 204, 304, 404, 504 and the posterior intraocular lens 102, 202, 302, 402, 502 otherwise. The degree of separation of the two intraocular lenses during accommodative reflex can contribute to a change in optical or dioptric power of the combined lens system.

The optical power or diopter distribution may be shared between the two lenses in each pair of lenses 102-104, 202-204, 302-304, 402-404, 502-504 in any suitable manner. Depending on the implementation, only one lens of each pair may provide a desired optical or dioptric power, or both lenses of each pair may collectively provide a desired optical or dioptric power. For example, the anterior intraocular lens 104, 204, 304, 404, 504 in each accommodating intraocular lens 100, 200, 300, 400, 500 may contribute all of the desired optical or dioptric power, and the posterior intraocular lens 102, 202, 302, 402, 502 can be neutral (plano in power). As another example, the posterior intraocular lens 102, 202, 302, 402, 502 in each accommodating intraocular lens 100, 200, 300, 400, 500 may contribute part of the desired optical or dioptric power, and the anterior intraocular lens 104, 204, 304, 404, 504 in each accommodating intraocular lens 100, 200, 300, 400, 500 may contribute another part of the desired optical or dioptric power. In some cases, one or both lenses in each pair of lenses 102-104, 202-204, 302-304, 402-404, 502-504 could offer other or additional optical modifications. For instance, the anterior intraocular lens 104, 204, 304, 404, 504 in each accommodating intraocular lens 100, 200, 300, 400, 500 may additionally offer an aspheric anterior refractive surface correction contributing to additional gain in near vision acuity.

During accommodative reflex in the patient's eye, the optical lens 106, 206, 306, 406, 506 of the posterior intraocular lens 102, 202, 302, 402, 502 may move. Moreover, during accommodative reflex in the patient's eye, the optical lens 110, 210, 310, 410, 510 of the anterior intraocular lens 104, 204, 304, 404, 504 may change shape and/or change position, such as when the anterior intraocular lens 104, 204, 304, 404, 504 in each accommodating intraocular lens 100, 200, 300, 400, 500 bulges outward somewhat during accommodative reflex. The movement or bulging of the anterior intraocular lens 104, 204, 304, 404, 504 can increase separation between the anterior intraocular lens 104, 204, 304, 404, 504 and the associated posterior intraocular lens 102, 202, 302, 402, 502. This can increase the optical or dioptric power provided by the accommodating intraocular lens 100, 200, 300, 400, 500 during accommodative reflex. This can also result in a change in the focal length of the accommodating intraocular lens 100, 200, 300, 400, 500. As a result, each pair of intraocular lenses 102-104, 202-204, 302-304, 402-404, 502-504 can function together as a combined unit to create an accommodative lens system.

Although FIGS. 1A through 5D illustrate examples of accommodating intraocular lenses having dual-lens systems supporting myopic shift in refraction during accommodative reflex, various changes may be made to FIGS. 1A through 5D. For example, the specific posterior intraocular lens used here may vary. A number of accommodating intraocular lenses are available today, and additional accommodating intraocular lenses are sure to be developed in the future. This disclosure is not necessarily limited to any specific form of the posterior intraocular lens. Also, the anterior intraocular lenses may be modified in a number of ways, and different anterior intraocular lenses may be used here. For instance, each of the anterior intraocular lenses 104, 204, 304, 404, 504 may include any suitable number and arrangement of haptics 112, 212, 312, 412, 512. In addition, a number of other features could be used at one or more locations of the accommodating intraocular lenses 100, 200, 300, 400, 500. As examples, each ridge of the haptics 112, 212, 312, 412, 512 may include a lip projecting inward from the ridge, and/or one or more drug-eluting materials or structures could be placed on the top, side, or bottom surface(s) of the accommodating intraocular lenses.

As noted above, a wide variety of anterior intraocular lenses may be used in a dual-lens system supporting myopic shift in refraction during accommodative reflex. The following now describes various examples of anterior intraocular lenses that may be used as the anterior intraocular lens 104, 204, 304, 404, 504 in any of the accommodating intraocular lenses 100, 200, 300, 400, 500 described above or other dual-lens system designed in accordance with the teachings of this disclosure. Note, however, that these are examples only and that other anterior intraocular lenses may be used in an accommodating intraocular lens.

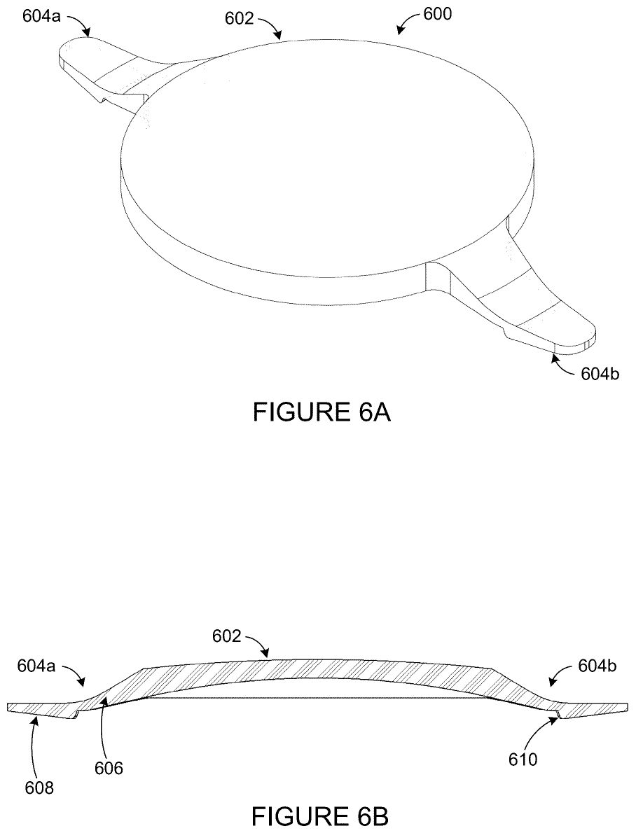

FIGS. 6A and 6B illustrate a first example anterior intraocular lens 600 that may be used in a dual-lens system supporting myopic shift in refraction during accommodative reflex according to this disclosure. For instance, the anterior intraocular lens 600 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 6A illustrates an oblique view of the anterior intraocular lens 600, and FIG. 6B illustrates a cross-sectional view through the middle of the anterior intraocular lens 600.

As shown in FIGS. 6A and 6B, the anterior intraocular lens 600 includes an optical lens 602 and multiple haptics 604a-604b. The optical lens 602 could be the same as or similar to the optical lens 110 described above. In this example, the haptics 604a-604b are formed by projections that extend from the sides of the optical lens 602. Each haptic 604a-604b includes an inner portion 606 that is connected to the optical lens 602 (or to a retaining ring in which the optical lens 602 is located) and an outer portion 608 that is connected to the inner portion 606, effectively forming “wings” extending from the optical lens 602.

In some cases, the outer portion 608 of each haptic 604a-604b may have a thickness that tapers towards the outer edge of the haptic 604a-604b. Also, in each haptic 604a-604b, the inner portion 606 projects outward and downward in this example, while the outer portion 608 projects outward and slightly upward in this example (although other forms could also be used). Each of the haptics 604a-604b also includes a ridge 610, and multiple ridges 610 of multiple haptics 604a-604b can be used to capture one or more edges of the underlying posterior intraocular lens or align the anterior intraocular lens 600 with the underlying posterior intraocular lens. This can help to center the anterior intraocular lens 600 on the underlying posterior intraocular lens.

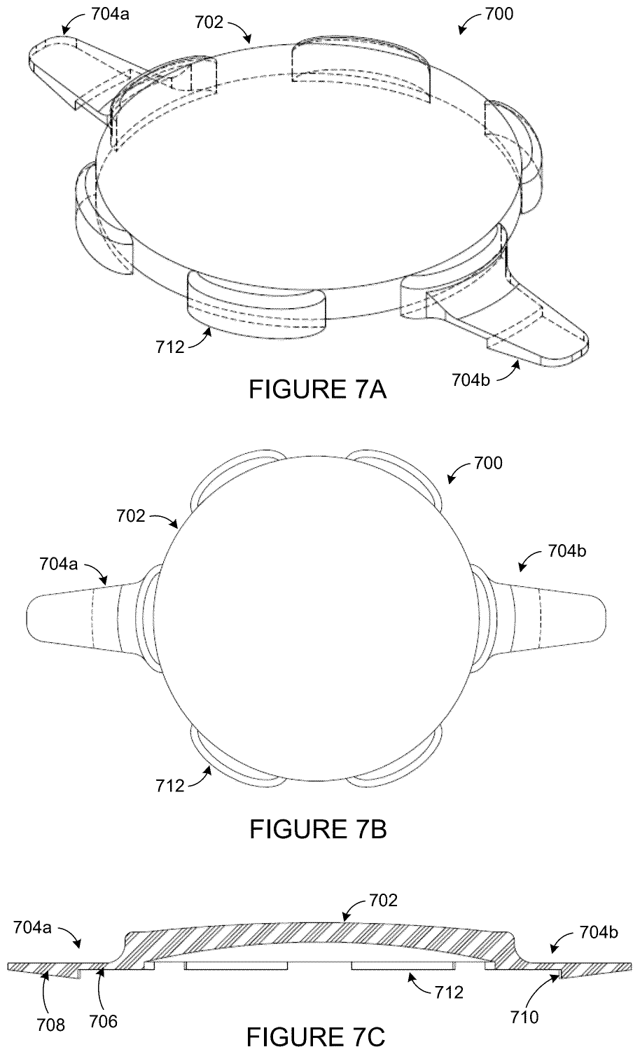

FIGS. 7A through 7C illustrate a second example anterior intraocular lens 700 that may be used in a dual-lens system supporting myopic shift in refraction during accommodative reflex according to this disclosure. For instance, the anterior intraocular lens 700 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 7A illustrates an oblique view of the anterior intraocular lens 700, FIG. 7B illustrates a top view of the anterior intraocular lens 700, and FIG. 7C illustrates a cross-sectional view through the middle of the anterior intraocular lens 700.

As shown in FIGS. 7A through 7C, the anterior intraocular lens 700 includes an optical lens 702 and multiple haptics 704a-704b. The optical lens 702 could be the same as or similar to the optical lens 110 described above. In this example, the haptics 704a-704b are formed by projections that extend from the sides of the optical lens 702. Each haptic 704a-704b includes an inner portion 706 that is connected to the optical lens 702 (or to a retaining ring in which the optical lens 702 is located) and an outer portion 708 that is connected to the inner portion 706.

In some cases, the outer portion 708 of each haptic 704a-704b may have a thickness that tapers towards the outer edge of the haptic 704a-704b. Also, in each haptic 704a-704b, the inner portion 706 and the outer portion 708 both project outward and straight in this example (although other forms could also be used). Each of the haptics 704a-704b also includes a ridge 710, and multiple ridges 710 of multiple haptics 704a-704b can be used to capture one or more edges of the underlying posterior intraocular lens or align the anterior intraocular lens 700 with the underlying posterior intraocular lens. This can help to center the anterior intraocular lens 700 on the underlying posterior intraocular lens.

In addition, the anterior intraocular lens 700 here includes multiple segments 712 located along the sides of the optical lens 702. The segments 712 denote projections from the optical lens 702, and at least some of the segments 712 could be coupled to the haptics 704a-704b (such as when ends of the haptics 704a-704b are embedded in the segments 712). The segments 712 extend downward so that the bottom surfaces of the segments 712 are located below the optical lens 702. As a result, the segments 712 keep the optical lens 702 separated from the underlying posterior intraocular lens. Depending on the shape of the posterior surface of the optical lens 702 and the shape of the anterior surface of the underlying posterior intraocular lens, this could elevate the optical lens 702 over an optical lens within the underlying posterior intraocular lens so that the optical lenses do not contact each other.

Each of the segments 712 could be formed from any suitable material(s) and in any suitable manner. For example, each segment 712 could represent a portion of the material(s) forming the optical lens 702 and therefore represent an extension of the optical lens 702 itself. However, this need not be the case. For instance, the optical lens 702 could be placed within a retaining ring that is integral with or attached to the segments 712, or the segments 712 could be secured to the optical lens 702 itself using adhesive or other suitable connecting mechanism. Each of the segments 712 could also have any suitable size, shape, and dimensions. For example, the segments 712 could be smaller or larger (relative to the other structures) than those shown in FIGS. 7A through 7C. As another example, the segments 712 could denote curved structures that leave small open areas between the segments 712 and the optical lens 702, or the segments 712 could be solid structures that leave no open areas between the segments 712 and the optical lens 702.

The ability to space the optical lens 702 away from the underlying posterior intraocular lens could provide various benefits. For example, elevating the optical lens 702 over the underlying posterior intraocular lens may allow for an increase in aqueous flow between the anterior surface of the underlying posterior intraocular lens and the posterior surface of the optical lens 702. An increased flow of aqueous between the lenses could help to reduce lens deposits on either or both of the lenses. Also, the presence of aqueous between the lenses can help to improve the optic or image quality of the combined lens system. In addition, by providing more space between the lenses, the anterior intraocular lens 700 could be used with a wider range of intraocular lenses that have varying anterior curvature surfaces, allowing the anterior intraocular lens 700 to be used with a wider range of intraocular lens models and powers.

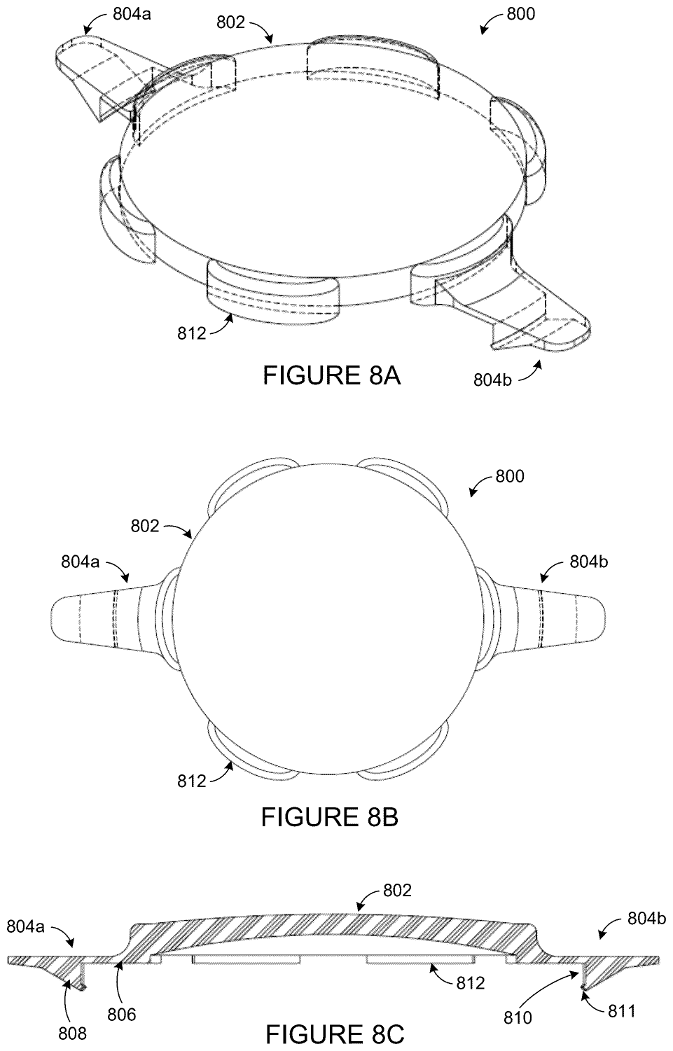

FIGS. 8A through 8C illustrate a third example anterior intraocular lens 800 that may be used in a dual-lens system supporting myopic shift in refraction during accommodative reflex according to this disclosure. For instance, the anterior intraocular lens 800 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 8A illustrates an oblique view of the anterior intraocular lens 800, FIG. 8B illustrates a top view of the anterior intraocular lens 800, and FIG. 8C illustrates a cross-sectional view through the middle of the anterior intraocular lens 800.

As shown in FIGS. 8A through 8C, the anterior intraocular lens 800 has various components that are the same as or similar to those forming the anterior intraocular lens 700. For example, the anterior intraocular lens 800 includes an optical lens 802 and multiple haptics 804a-804b. In this example, the haptics 804a-804b are formed by projections that extend from the sides of the optical lens 802. Each haptic 804a-804b includes an inner portion 806 that is connected to the optical lens 802 (or to a retaining ring in which the optical lens 802 is located) and an outer portion 808 that is connected to the inner portion 806. Each of the haptics 804a-804b also includes a ridge 810, and multiple ridges 810 of multiple haptics 804a-804b can be used to capture one or more edges of the underlying posterior intraocular lens or align the anterior intraocular lens 800 with the underlying posterior intraocular lens. In addition, the anterior intraocular lens 800 includes multiple segments 812 along the sides of the optical lens 802. The segments 812 extend downward so that the bottom surfaces of the segments 812 are located below the optical lens 802.

The haptics 804a-804b in this example include thicker outer portions 808 with larger ridges 810 compared to the corresponding components of the anterior intraocular lens 700. This allows the haptics 804a-804b to be used with larger intraocular lenses. Moreover, each of the ridges 810 includes a lip 811 that can facilitate the capture of the underlying posterior intraocular lens or alignment of the anterior intraocular lens 800 with the underlying posterior intraocular lens. Each of the lips 811 denotes any suitable inward projection from the corresponding ridge 810.

Again, the segments 812 help to keep the optical lens 802 separated from the underlying posterior intraocular lens. Depending on the shape of the posterior surface of the optical lens 802 and the shape of the anterior surface of the underlying posterior intraocular lens, this could elevate the optical lens 802 over an optical lens within the underlying posterior intraocular lens so that the optical lenses do not contact each other. The ability to space the optical lens 802 away from the underlying posterior intraocular lens could provide various benefits, such as those described above with respect to the anterior intraocular lens 700.

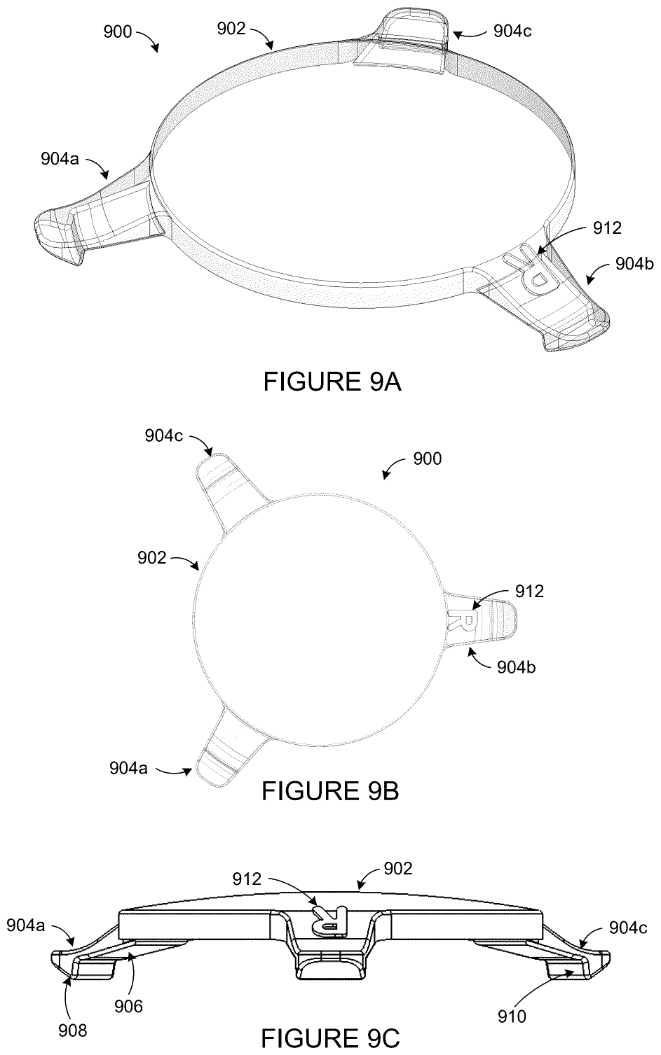

FIGS. 9A through 9C illustrate a fourth example anterior intraocular lens 900 that may be used in a dual-lens system supporting myopic shift in refraction during accommodative reflex according to this disclosure. For instance, the anterior intraocular lens 900 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 9A illustrates an oblique view of the anterior intraocular lens 900, FIG. 9B illustrates a top view of the anterior intraocular lens 900, and FIG. 9C illustrates a side view of the anterior intraocular lens 900.

As shown in FIGS. 9A through 9C, the anterior intraocular lens 900 has various components that are the same as or similar to those forming the anterior intraocular lens 600. For example, the anterior intraocular lens 900 includes an optical lens 902 and multiple haptics 904a-904c. In this example, the haptics 904a-904c are formed by projections that extend from the sides of the optical lens 902. Each haptic 904a-904c includes an inner portion 906 that is connected to the optical lens 902 (or to a retaining ring in which the optical lens 902 is located) and an outer portion 908 that is connected to the inner portion 906. Each of the haptics 904a-904c also includes a ridge 910, and multiple ridges 910 of multiple haptics 904a-904c can be used to capture one or more edges of the underlying posterior intraocular lens or align the anterior intraocular lens 900 with the underlying posterior intraocular lens. While not shown here, at least one of the ridges 910 may include a lip, which can be the same as or similar to the lip 811 and which can facilitate the capture and retention of the underlying posterior intraocular lens.

In some cases, the outer portion 908 of each haptic 904a-904c may have a thickness that tapers towards the outer edge of the haptic 904a-904c. Also, in each haptic 904a-904c, the inner portion 906 projects outward and downward in this example, while the outer portion 908 projects outward and slightly upward in this example (although other forms could also be used). While three haptics 904a-904c are shown here, other numbers of haptics could also be used. In some embodiments, the haptics 904a-904c can be positioned at an even spacing of 120°.

Also in this example, at least one alignment marking 912 may be provided on one or more of the haptics 904a-904c. The alignment marking 912 may be the same as or similar to the alignment marking 514 described above. In this particular example, the alignment marking 912 represents the letter “R”, although any other suitable marking(s) may be used here in any suitable location(s). The alignment marking 912 can be used to identify the proper orientation of the anterior intraocular lens 900, such as by identifying the haptic 904b that is to be positioned on the right side of the anterior intraocular lens 900 (from the perspective of the surgeon or other medical personnel) after implantation.

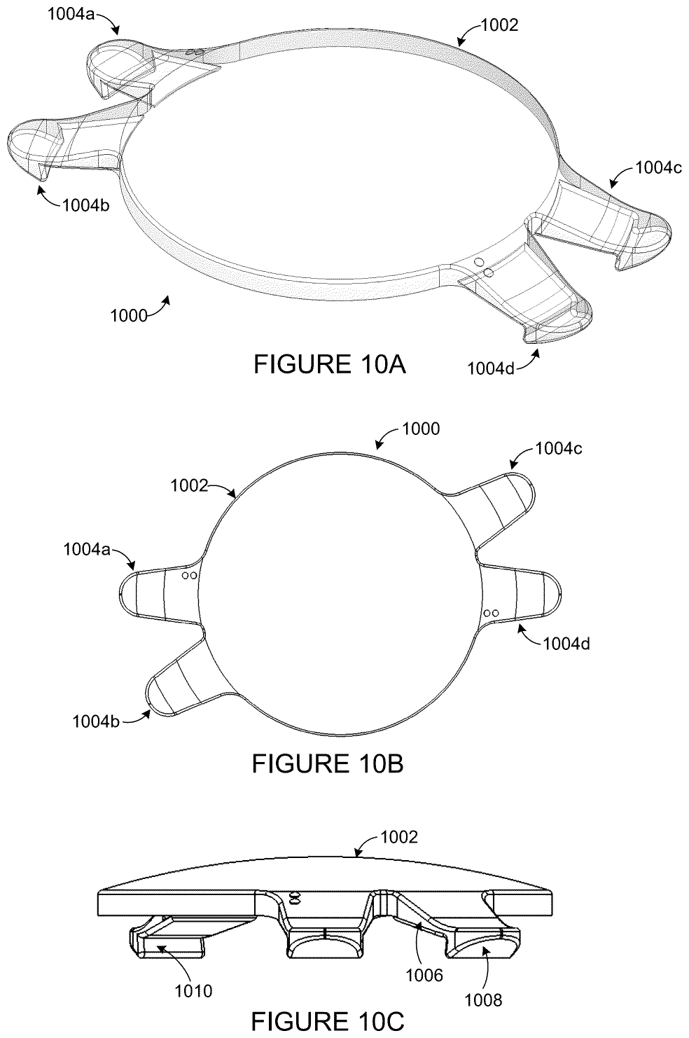

FIGS. 10A through 10C illustrate a fifth example anterior intraocular lens 1000 that may be used in a dual-lens system supporting myopic shift in refraction during accommodative reflex according to this disclosure. For instance, the anterior intraocular lens 1000 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 10A illustrates an oblique view of the anterior intraocular lens 1000, FIG. 10B illustrates a top view of the anterior intraocular lens 1000, and FIG. 10C illustrates a side view of the anterior intraocular lens 1000.

As shown in FIGS. 10A through 10C, the anterior intraocular lens 1000 has various components that are the same as or similar to those forming the anterior intraocular lens 900. For example, the anterior intraocular lens 1000 includes an optical lens 1002 and multiple haptics 1004a-1004d. In this example, the haptics 1004a-1004d are formed by projections that extend from the sides of the optical lens 1002. Each haptic 1004a-1004d includes an inner portion 1006 that is connected to the optical lens 1002 (or to a retaining ring in which the optical lens 1002 is located) and an outer portion 1008 that is connected to the inner portion 1006. Each of the haptics 1004a-1004d also includes a ridge 1010, and multiple ridges 1010 of multiple haptics 1004a-1004d can be used to capture one or more edges of the underlying posterior intraocular lens or align the anterior intraocular lens 1000 with the underlying posterior intraocular lens. While not shown here, at least one of the ridges 1010 may include a lip, which can be the same as or similar to the lip 811 and which can facilitate the capture and retention of the underlying posterior intraocular lens.

In some cases, the outer portion 1008 of each haptic 1004a-1004d may have a thickness that tapers towards the outer edge of the haptic 1004a-1004d. Also, in each haptic 1004a-1004d, the inner portion 1006 projects outward and downward in this example, while the outer portion 1008 projects outward and slightly upward in this example (although other forms could also be used). While four haptics 1004a-1004d are shown here in two groups on opposite sides of the optical lens 1002, other numbers and arrangements of haptics could also be used.

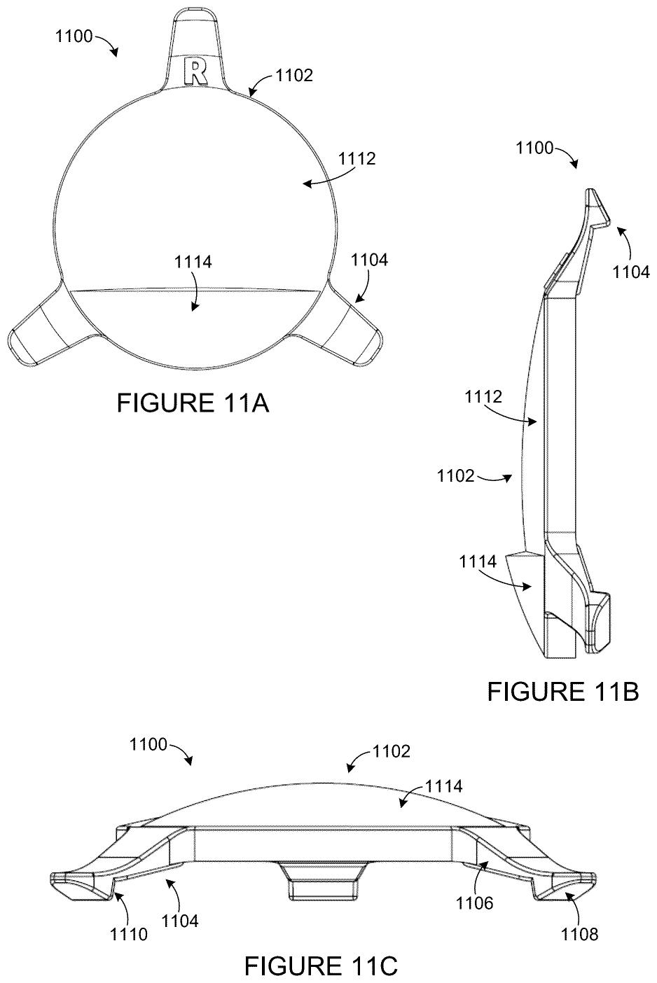

FIGS. 11A through 11C illustrate a sixth example anterior intraocular lens 1100 that may be used in a dual-lens system supporting myopic shift in refraction during accommodative reflex according to this disclosure. For instance, the anterior intraocular lens 1100 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 11A illustrates a top view of the anterior intraocular lens 1100, FIG. 11B illustrates a side view of the anterior intraocular lens 1100, and FIG. 11C illustrates a bottom view of the anterior intraocular lens 1100.

As shown in FIGS. 11A through 11C, the anterior intraocular lens 1100 has various components that are the same as or similar to those forming the anterior intraocular lens 900. For example, the anterior intraocular lens 1100 includes an optical lens 1102 and multiple haptics 1104. In this example, the haptics 1104 are formed by projections that extend from the sides of the optical lens 1102. Each haptic 1104 includes an inner portion 1106 that is connected to the optical lens 1102 (or to a retaining ring in which the optical lens 1102 is located) and an outer portion 1108 that is connected to the inner portion 1106. Each of the haptics 1104 also includes a ridge 1110, and multiple ridges 1110 of multiple haptics 1104 can be used to capture one or more edges of the underlying posterior intraocular lens or align the anterior intraocular lens 1100 with the underlying posterior intraocular lens. While not shown here, at least one of the ridges 1110 may include a lip, which can be the same as or similar to the lip 811 and which can facilitate the capture and retention of the underlying posterior intraocular lens.

In some cases, the outer portion 1108 of each haptic 1104a-1104c may have a thickness that tapers towards the outer edge of the haptic 1104a-1104c. Also, in each haptic 1104a-1104c, the inner portion 1106 projects outward and downward in this example, while the outer portion 1108 projects outward and slightly upward in this example (although other forms could also be used). While three haptics 1104 are shown here, other numbers and arrangements of haptics could also be used.

In this example, the optical lens 1102 of the anterior intraocular lens 1100 includes a first lens portion 1112 and a second lens portion 1114. The two portions 1112-1114 of the optical lens 1102 may be used to provide different levels of optical magnification. In this example, for instance, the first lens portion 1112 may provide a specified amount of magnification (or possibly little or no magnification), and the second lens portion 1114 may provide a higher specified amount of magnification. In some embodiments, the second lens portion 1114 may represent a “full seg” of added magnification compared to the first lens portion 1112. Note that the amount of magnification provided by each of the lens portions 1112-1114 may or may not be based on any residual refractive error in a patient's eye. Also note that while the lens portion 1114 with additional magnification is placed at the bottom of the optical lens 1102, a lens portion with additional magnification may be placed in any other suitable location(s) or along any other suitable axis or axes of the optical lens 1102. Further note that the sizes and shapes of the lens portions 1112-1114 can vary as needed or desired.

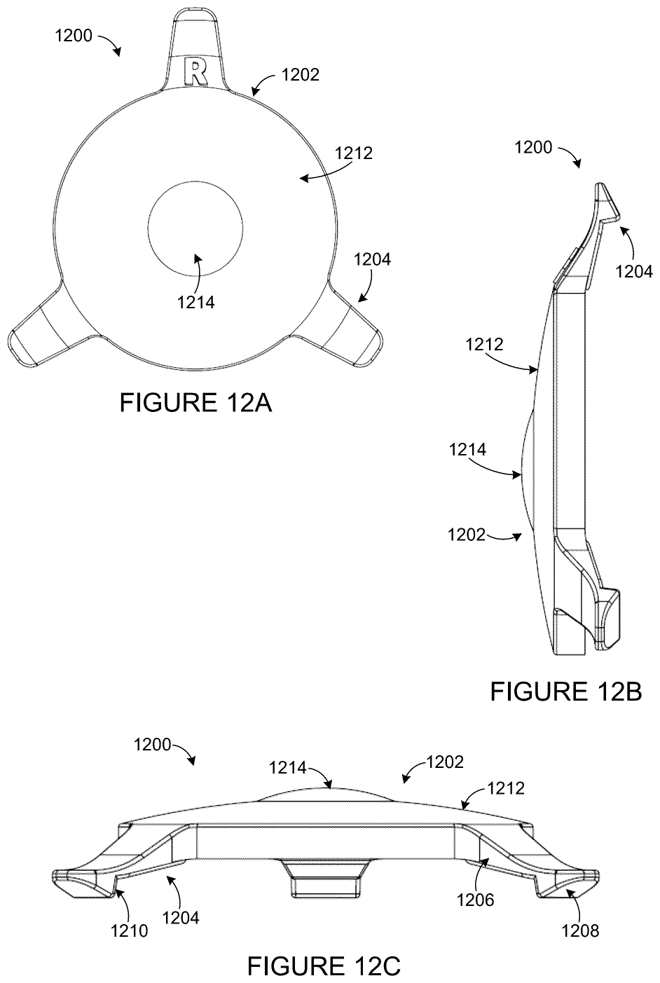

FIGS. 12A through 12C illustrate a seventh example anterior intraocular lens 1200 that may be used in a dual-lens system supporting myopic shift in refraction during accommodative reflex according to this disclosure. For instance, the anterior intraocular lens 1200 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 12A illustrates a top view of the anterior intraocular lens 1200, FIG. 12B illustrates a side view of the anterior intraocular lens 1200, and FIG. 12C illustrates a bottom view of the anterior intraocular lens 1200.

As shown in FIGS. 12A through 12C, the anterior intraocular lens 1200 has various components that are the same as or similar to those forming the anterior intraocular lens 1100. For example, the anterior intraocular lens 1200 includes an optical lens 1202 and multiple haptics 1204. In this example, the haptics 1204 are formed by projections that extend from the sides of the optical lens 1202. Each haptic 1204 includes an inner portion 1206 that is connected to the optical lens 1202 (or to a retaining ring in which the optical lens 1202 is located) and an outer portion 1208 that is connected to the inner portion 1206. Each of the haptics 1204 also includes a ridge 1210, and multiple ridges 1210 of multiple haptics 1204 can be used to capture one or more edges of the underlying posterior intraocular lens or align the anterior intraocular lens 1200 with the underlying posterior intraocular lens. While not shown here, at least one of the ridges 1210 may include a lip, which can be the same as or similar to the lip 811 and which can facilitate the capture and retention of the underlying posterior intraocular lens.

In some cases, the outer portion 1208 of each haptic 1204a-1204c may have a thickness that tapers towards the outer edge of the haptic 1204a-1204c. Also, in each haptic 1204a-1204c, the inner portion 1206 projects outward and downward in this example, while the outer portion 1208 projects outward and slightly upward in this example (although other forms could also be used). While three haptics 1204 are shown here, other numbers and arrangements of haptics could also be used.

In this example, the optical lens 1202 of the anterior intraocular lens 1200 includes a first lens portion 1212 and a second lens portion 1214. The two portions 1212-1214 of the optical lens 1202 may be used to provide different levels of optical magnification. In this example, for instance, the first lens portion 1212 may provide a specified amount of magnification (or possibly little or no magnification), and the second lens portion 1214 may provide a higher specified amount of magnification. Note that the amount of magnification provided by each of the lens portions 1212-1214 may or may not be based on any residual refractive error in a patient's eye. Also note that while the lens portion 1214 with additional magnification is placed in the center of the optical lens 1202, a lens portion with additional magnification may be placed in any other suitable location(s) or along any other suitable axis or axes of the optical lens 1202. Further note that the sizes and shapes of the lens portions 1212-1214 can vary as needed or desired.

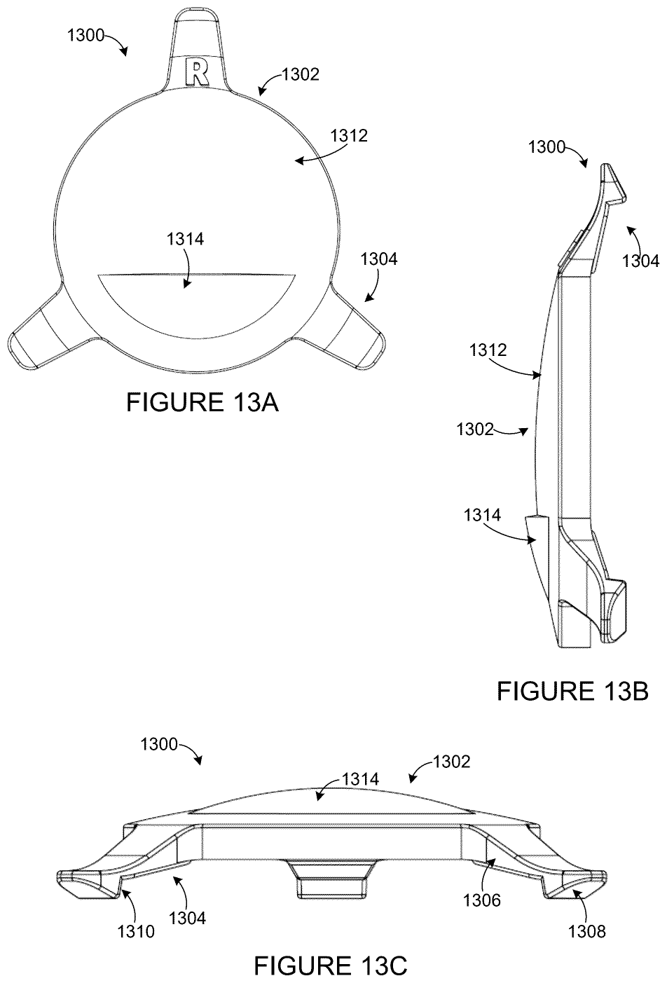

FIGS. 13A through 13C illustrate an eighth example anterior intraocular lens 1300 that may be used in a dual-lens system supporting myopic shift in refraction during accommodative reflex according to this disclosure. For instance, the anterior intraocular lens 1300 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 13A illustrates a top view of the anterior intraocular lens 1300, FIG. 13B illustrates a side view of the anterior intraocular lens 1300, and FIG. 13C illustrates a bottom view of the anterior intraocular lens 1300.

As shown in FIGS. 13A through 13C, the anterior intraocular lens 1300 has various components that are the same as or similar to those forming the anterior intraocular lens 1100. For example, the anterior intraocular lens 1300 includes an optical lens 1302 and multiple haptics 1304. In this example, the haptics 1304 are formed by projections that extend from the sides of the optical lens 1302. Each haptic 1304 includes an inner portion 1306 that is connected to the optical lens 1302 (or to a retaining ring in which the optical lens 1302 is located) and an outer portion 1308 that is connected to the inner portion 1306. Each of the haptics 1304 also includes a ridge 1310, and multiple ridges 1310 of multiple haptics 1304 can be used to capture one or more edges of the underlying posterior intraocular lens or align the anterior intraocular lens 1300 with the underlying posterior intraocular lens. While not shown here, at least one of the ridges 1310 may include a lip, which can be the same as or similar to the lip 811 and which can facilitate the capture and retention of the underlying posterior intraocular lens.

In some cases, the outer portion 1308 of each haptic 1304a-1304c may have a thickness that tapers towards the outer edge of the haptic 1304a-1304c. Also, in each haptic 1304a-1304c, the inner portion 1306 projects outward and downward in this example, while the outer portion 1308 projects outward and slightly upward in this example (although other forms could also be used). While three haptics 1304 are shown here, other numbers and arrangements of haptics could also be used.

In this example, the optical lens 1302 of the anterior intraocular lens 1300 includes a first lens portion 1312 and a second lens portion 1314. The two portions 1312-1314 of the optical lens 1302 may be used to provide different levels of optical magnification. In this example, for instance, the first lens portion 1312 may provide a specified amount of magnification (or possibly little or no magnification), and the second lens portion 1314 may provide a higher specified amount of magnification. In some embodiments, the second lens portion 1314 may represent a “small seg” of added magnification compared to the first lens portion 1312. Note that the amount of magnification provided by each of the lens portions 1312-1314 may or may not be based on any residual refractive error in a patient's eye. Also note that while the lens portion 1314 with additional magnification is placed near the bottom of the optical lens 1302, a lens portion with additional magnification may be placed in any other suitable location(s) or along any other suitable axis or axes of the optical lens 1302. Further note that the sizes and shapes of the lens portions 1312-1314 can vary as needed or desired.

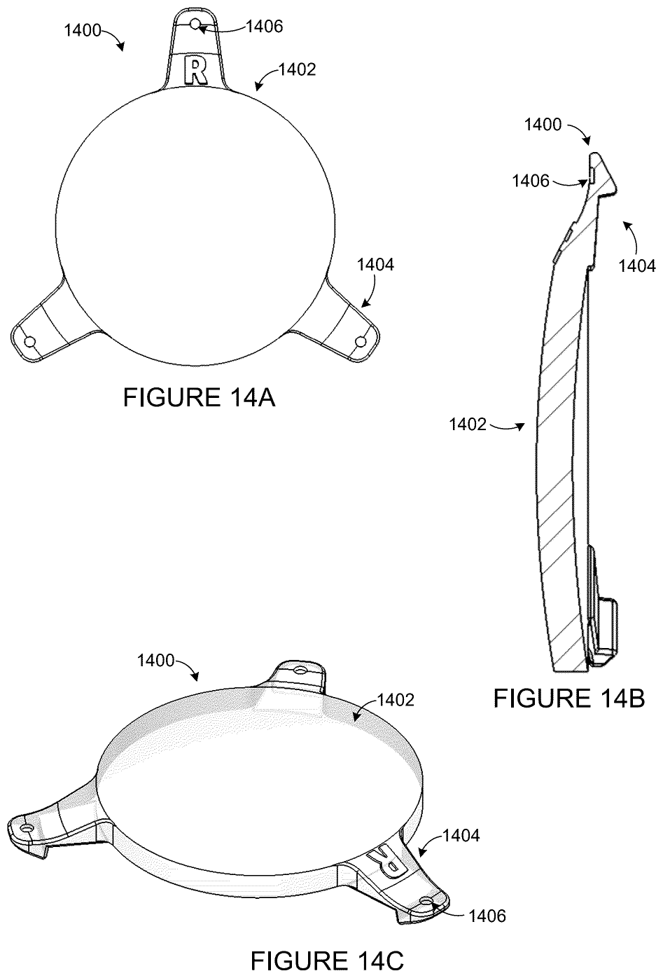

FIGS. 14A through 14C illustrate a ninth example anterior intraocular lens 1400 that may be used in a dual-lens system supporting myopic shift in refraction during accommodative reflex according to this disclosure. For instance, the anterior intraocular lens 1400 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 14A illustrates a top view of the anterior intraocular lens 1400, FIG. 14B illustrates a cross-sectional view of the anterior intraocular lens 1400, and FIG. 14C illustrates a perspective view of the anterior intraocular lens 1400.

As shown in FIGS. 14A through 14C, the anterior intraocular lens 1400 has various components that are the same as or similar to those forming anterior intraocular lenses described above. For example, the anterior intraocular lens 1400 includes an optical lens 1402 and multiple haptics 1404. In this example, the haptics 1404 are formed by projections that extend from the sides of the optical lens 1402. Each haptic 1404 may include an inner portion that is connected to the optical lens 1402 (or to a retaining ring in which the optical lens 1402 is located) and an outer portion that is connected to the inner portion. Each of the haptics 1404 may also include a ridge, and multiple ridges of multiple haptics 1404 can be used to capture one or more edges of the underlying posterior intraocular lens or align the anterior intraocular lens 1400 with the underlying posterior intraocular lens. While not shown here, at least one of the ridges may include a lip, which can be the same as or similar to the lip 811 and which can facilitate the capture and retention of the underlying posterior intraocular lens.

In some cases, an outer portion of each haptic 1404a-1404c may have a thickness that tapers towards the outer edge of the haptic 1404a-1404c. Also, in each haptic 1404a-1404c, an inner portion projects outward and downward in this example, while the outer portion projects outward and slightly upward in this example (although other forms could also be used). While three haptics 1404 are shown here, other numbers and arrangements of haptics could also be used.

In this example, each of the haptics 1404 includes a divot or hole 1406, which is positioned along the anterior surface of the haptic 1404 and can extend partially or completely through the haptic 1404. In the illustrated example, the divot or hole 1406 extends partially through the haptic 1404. Each divot or hole 1406 can be used by a surgeon or other personnel to manipulate the haptic 1404, the anterior intraocular lens as a whole, or the accommodating intraocular lens as a whole. For instance, the surgeon or other personnel may use an intraocular lens manipulator, like a Lewicky hook, a Sinskey hook, or other surgical tool, to manipulate the haptic 1404, anterior intraocular lens, or accommodating intraocular lens. Each divot or hole 1406 may have any suitable size, shape, and dimensions.

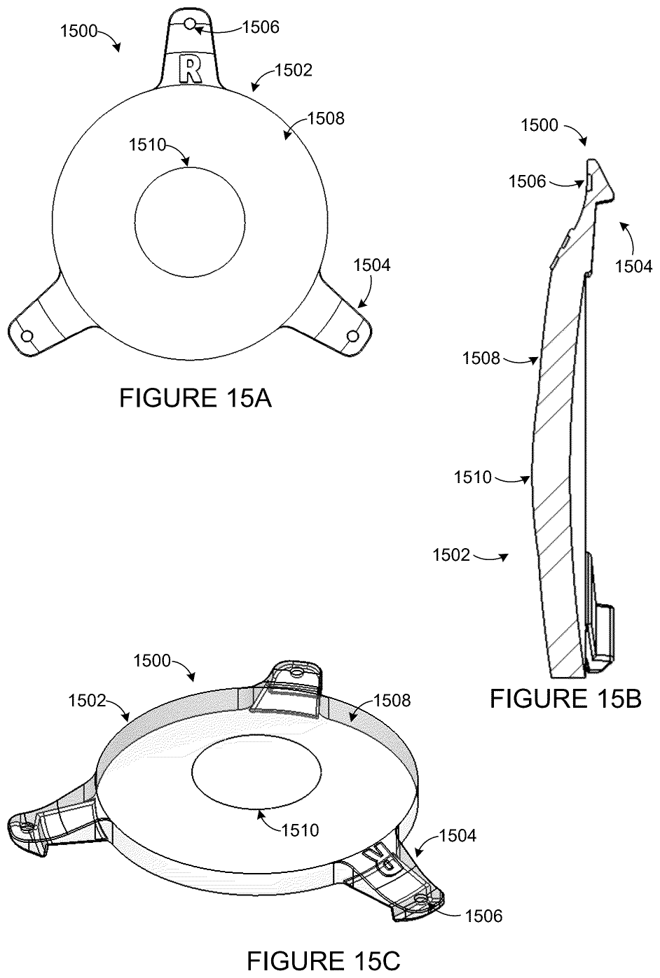

FIGS. 15A through 15C illustrate a tenth example anterior intraocular lens 1500 that may be used in a dual-lens system supporting myopic shift in refraction during accommodative reflex according to this disclosure. For instance, the anterior intraocular lens 1500 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 15A illustrates a top view of the anterior intraocular lens 1500, FIG. 15B illustrates a cross-sectional view of the anterior intraocular lens 1500, and FIG. 15C illustrates a perspective view of the anterior intraocular lens 1500.

As shown in FIGS. 15A through 15C, the anterior intraocular lens 1500 has various components that are the same as or similar to those forming anterior intraocular lenses described above. For example, the anterior intraocular lens 1500 includes an optical lens 1502 and multiple haptics 1504. The haptics 1504 are formed by projections that extend from the sides of the optical lens 1502. Each haptic 1504 may include an inner portion that is connected to the optical lens 1502 (or to a retaining ring in which the optical lens 1502 is located) and an outer portion that is connected to the inner portion. Each of the haptics 1504 may also include a ridge, and multiple ridges of multiple haptics 1504 can be used to capture one or more edges of the underlying posterior intraocular lens or align the anterior intraocular lens 1500 with the underlying posterior intraocular lens. While not shown here, at least one of the ridges may include a lip, which can be the same as or similar to the lip 811 and which can facilitate the capture and retention of the underlying posterior intraocular lens.

In some cases, the outer portion of each haptic 1504a-1504c may have a thickness that tapers towards the outer edge of the haptic 1504a-1504c. Also, in each haptic 1504a-1504c, an inner portion projects outward and downward in this example, while an outer portion projects outward and slightly upward in this example (although other forms could also be used). While three haptics 1504 are shown here, other numbers and arrangements of haptics could also be used.

In this example, each of the haptics 1504 includes a divot or hole 1506, which is positioned along the anterior surface of the haptic 1504 and can extend partially or completely through the haptic 1504. In the illustrated example, the divot or hole 1506 extends partially through the haptic 1504. Each divot or hole 1506 can be used by a surgeon or other personnel to manipulate the haptic 1504, the anterior intraocular lens as a whole, or the accommodating intraocular lens as a whole. For instance, the surgeon or other personnel may use an intraocular lens manipulator, like a Lewicky hook, a Sinskey hook, or other surgical tool, to manipulate the haptic 1504, anterior intraocular lens, or accommodating intraocular lens. Each divot or hole 1506 may have any suitable size, shape, and dimensions.

In the illustrated example, the optical lens 1502 of the anterior intraocular lens 1500 includes a first lens portion 1508 and a second lens portion 1510. The two portions 1508-1510 of the optical lens 1502 may be used to provide different levels of optical magnification. In this example, for instance, the first lens portion 1508 may provide a specified amount of magnification (or possibly little or no magnification), and the second lens portion 1510 may provide a higher specified amount of magnification. In some embodiments, the second lens portion 1510 may represent a “small seg” of added magnification compared to the first lens portion 1508. Note that the amount of magnification provided by each of the lens portions 1508-1510 may or may not be based on any residual refractive error in a patient's eye. Also note that while the lens portion 1510 with additional magnification is placed in the center of the optical lens 1502, a lens portion with additional magnification may be placed in any other suitable location(s) or along any other suitable axis or axes of the optical lens 1502. Further note that the sizes and shapes of the lens portions 1508-1510 can vary as needed or desired.

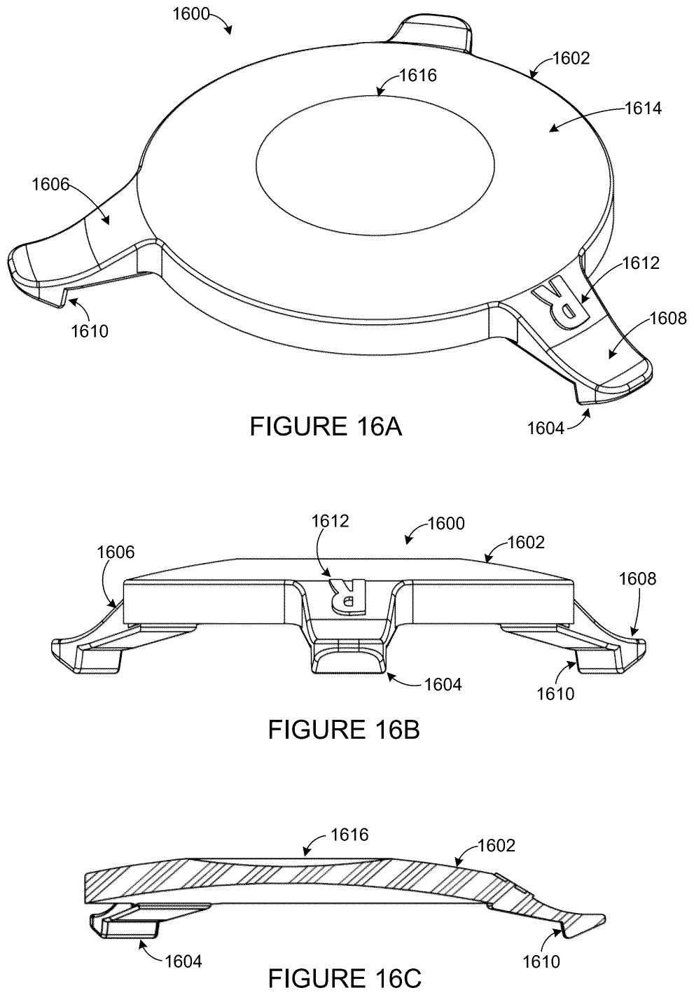

FIGS. 16A through 16C illustrate an eleventh example of an anterior intraocular lens 1600 that may be used in a dual-lens system supporting myopic shift in refraction during accommodative reflex according to this disclosure. For instance, the anterior intraocular lens 1600 may be used as the anterior intraocular lens 104, 204, 304, 404, 504 in any of the accommodating intraocular lenses 100, 200, 300, 400, 500 described above or other dual-lens system designed in accordance with the teachings of this disclosure. In particular, FIG. 16A illustrates an oblique view of the anterior intraocular lens 1600, FIG. 16B illustrates a side view of the anterior intraocular lens 1600, and FIG. 16C illustrates a cross-sectional view of the anterior intraocular lens 1600.

As shown in FIGS. 16A through 16C, the anterior intraocular lens 1600 has various components that are the same as or similar to those forming anterior intraocular lenses described above. For example, the anterior intraocular lens 1600 includes an optical lens 1602 and multiple haptics 1604. The haptics 1604 are formed by projections that extend from the sides of the optical lens 1602. Each haptic 1604 may include an inner portion 1606 that is connected to the optical lens 1602 (or to a retaining ring in which the optical lens 1602 is located) and an outer portion 1608 that is connected to the inner portion 1606. Each of the haptics 1604 may also include a ridge 1610, and multiple ridges 1610 of multiple haptics 1604 can be used to capture one or more edges of the underlying posterior intraocular lens or align the anterior intraocular lens 1600 with the underlying posterior intraocular lens. While not shown here, at least one of the ridges 1610 may include a lip, which can be the same as or similar to the lip 811 and which can facilitate the capture and retention of the underlying posterior intraocular lens.

In some cases, the outer portion 1608 of each haptic 1604 may have a thickness that tapers towards the outer edge of the haptic 1604. Also, in each haptic 1604, the inner portion 1606 projects outward and downward in this example, while the outer portion 1608 projects outward and slightly upward in this example (although other forms could also be used). While three haptics 1604 are shown here, other numbers and arrangements of haptics could also be used. At least one alignment marking 1612 may be provided on one or more of the haptics 1604. The alignment marking 1612 may be the same as or similar to the alignment markings 514, 912 described above.

In the illustrated example, the optical lens 1602 of the anterior intraocular lens 1600 includes a first lens portion 1614 and a second lens portion 1616. The two portions 1614-1616 of the optical lens 1602 may be used to provide different levels of optical magnification. In this example, for instance, the first lens portion 1614 may provide a specified amount of magnification (or possibly little or no magnification), and the second lens portion 1616 may provide a higher specified amount of magnification. Note that the amount of magnification provided by each of the lens portions 1614-1616 may or may not be based on any residual refractive error in a patient's eye. Also note that while the lens portion 1616 with additional magnification is placed in the center of the optical lens 1602, a lens portion with additional magnification may be placed in any other suitable location(s) or along any other suitable axis or axes of the optical lens 1602. Further note that the sizes and shapes of the lens portions 1614-1616 can vary as needed or desired.

In this particular example, the second lens portion 1616 (unlike similarly-situated lens portions described above) includes anterior and posterior surfaces that are both concave. This type of design may allow the second lens portion 1616 to provide a high minus power magnification. The first lens portion 1614 may provide a different amount of magnification or possibly little or no magnification. In some cases, the amount of high minus power magnification provided by the second lens portion 1616 can be controlled by altering the shape of one or more of its anterior and posterior surfaces, such as by making the anterior surface of the second lens portion 1616 more or less concave. In some cases, the second lens portion 1616 may provide an optical power between −5 diopters and −25 diopters (although these are example values only).

The various anterior intraocular lenses described above could have any suitable size, shape, and dimensions. For example, the anterior intraocular lenses could be made available in a range of diameters from about 4 mm to about 15 mm. Also, the anterior intraocular lenses could be made available with varying base curvatures for their optical lenses. Of course, an anterior intraocular lens could also be custom designed for a particular patient's eye, such as to provide a desired vision correction or other vision adjustment in the particular patient's eye. Additional details of various intraocular pseudophakic contact lenses that can be used as anterior intraocular lenses can be found in U.S. Patent Publication No. 2022/0280283 and U.S. Pat. No. 11,864,991 (both of which are hereby incorporated by reference in their entirety).

Although FIGS. 6A through 16C illustrate examples of anterior intraocular lenses that may be used in a dual-lens system supporting myopic shift in refraction during accommodative reflex, various changes may be made to FIGS. 6A through 16C. For example, any suitable combination of features shown in FIGS. 6A through 16C could be used together in a single anterior intraocular lens, whether or not that specific combination of features is shown in the figures or described above. As a particular example, any of the anterior intraocular lenses shown in FIGS. 6A through 16C could include one or more lips on its ridge(s), one or more divots in its haptic(s), one or more alignment markings, an optical lens having different lens portions with different magnifications, and/or convex and/or concave surfaces as anterior and posterior surfaces of its optical lens. Also, each anterior intraocular lens could include any suitable number of each component shown in any of the figures. While the figures have shown the anterior intraocular lenses as having two, three, or four haptics (some at an even spacing of 120° or) 180°, any number of haptics (with or without associated ridges, lips, or other structures) could be used. Further, the forms of the haptics shown here are examples only, and any other suitable structures could be used. In addition, a number of other features could be used at one or more locations of the anterior intraocular lenses. For instance, one or more drug-eluting materials could be placed on top, side, or bottom surfaces of the anterior intraocular lenses or the accommodating intraocular lenses.

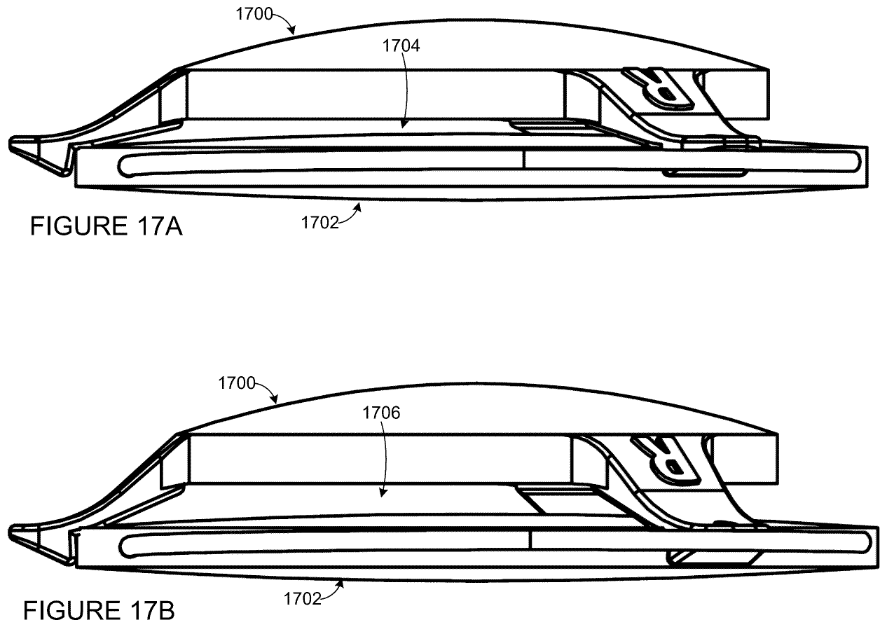

FIGS. 17A and 17B illustrate example operation of an accommodating intraocular lens having a dual-lens system supporting myopic shift in refraction during accommodative reflex according to this disclosure. In this example, the accommodating intraocular lens includes an anterior intraocular lens 1700 and a posterior intraocular lens 1702. The anterior intraocular lens 1700 may represent any of the anterior intraocular lenses described above or any anterior intraocular lenses described in accordance with the teachings of this disclosure. The posterior intraocular lens 1702 may represent any of the posterior intraocular lenses described above or any posterior intraocular lenses described in accordance with the teachings of this disclosure.

In the state shown in FIG. 17A, there is an absence of accommodative reflex in a patient's eye. In other words, the patient is not attempting to focus at near distance. In this state, the ciliary muscles of the patient's eye are not attempting to change the shape of the previously-removed crystalline lens of the patient's eye. During this time, the posterior intraocular lens 1702 can remain in a substantially fixed position within the patient's eye. Since the anterior intraocular lens 1700 is attached to the posterior intraocular lens 1702, this also keeps the anterior intraocular lens 1700 in a substantially fixed position within the patient's eye in front of the posterior intraocular lens 1702 by a defined gap 1704. Note that while the gap 1704 here is shown as having a non-zero thickness, the gap 1704 may be non-existent in other embodiments.

In the state shown in FIG. 17B, there is accommodative reflex occurring in the patient's eye. During accommodative reflex, the ciliary muscles of the patient's eye are attempting to change the shape of the previously-removed crystalline lens of the patient's eye. Since the crystalline lens has been replaced by the posterior intraocular lens 1702, this may instead result in forward movement of the posterior intraocular lens 1702 within the patient's eye. Again, since the anterior intraocular lens 1700 is attached to the posterior intraocular lens 1702, this also moves the anterior intraocular lens 1700 forward within the patient's eye.

As described above, the posterior intraocular lens 1702 may be formed using at least one more rigid material, such as acrylic or stiffer silicone. The anterior intraocular lens 1700 may be formed using at least one more flexible material, such as more flexible silicone. During accommodative reflex, the ciliary muscles in the patient's eye contract and encroach the vitreous cavity chamber of the eye, which increases vitreous pressure. This increase in pressure can cause the lens system formed by the lenses 1700-1702 to shift forward within the patient's eye. This also allows the anterior intraocular lens 1700 to flex forward more than the more-rigid posterior intraocular lens 1702. Effectively, this causes the anterior intraocular lens 1700 to change shape and/or change position, such as by bulging forward during accommodative reflex. In this case, for instance, the optical lens of the anterior intraocular lens 1700 may move forwards (anteriorly) somewhat, while the haptics of the anterior intraocular lens 1700 may bend backwards (posteriorly) more. This maintains the connection of the intraocular lenses 1700-1702 and causes the intraocular lenses 1700-1702 to separate. This creates a larger gap 1706 between the intraocular lenses 1700-1702, and the larger gap 1706 leads to an increase in the dioptric power of the accommodating intraocular lens formed by the intraocular lenses 1700-1702.

In this way, the anterior movement of the lens system and the increase in separation between the intraocular lenses 1700-1702 allow for an increase in near vision, meaning the patient is able to focus more clearly on nearby objects. When the accommodative reflex ends, the lens system can return to the state shown in FIG. 17A, during which the patient is able to focus more clearly on further objects. As a result, the accommodating intraocular lens can provide a larger depth of focus range for the patient.

Although FIGS. 17A and 17B illustrate one example of operation of an accommodating intraocular lens having a dual-lens system supporting myopic shift in refraction during accommodative reflex, various changes may be made to FIGS. 17A and 17B. For example, the intraocular lenses 1700-1702 may separate to a greater or lesser extent during accommodative reflex.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in this patent document should not be read as implying that any particular element, step, or function is an essential or critical element that must be included in the claim scope. Also, none of the claims is intended to invoke 35 U.S.C. § 112 (f) with respect to any of the appended claims or claim elements unless the exact words “means for” or “step for” are explicitly used in the particular claim, followed by a participle phrase identifying a function. Use of terms such as (but not limited to) “mechanism”, “module”, “device”, “unit”, “component”, “element”, “member”, “apparatus”, “machine”, “system”, “processor”, “processing device”, or “controller” within a claim is understood and intended to refer to structures known to those skilled in the relevant art, as further modified or enhanced by the features of the claims themselves, and is not intended to invoke 35 U.S.C. § 112 (f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An accommodating intraocular lens system comprising:
a posterior intraocular lens comprising a first optical lens and first haptics; and
an anterior intraocular lens comprising a second optical lens and second haptics, the second haptics attached to the posterior intraocular lens;
wherein the posterior intraocular lens is configured to remain in a substantially fixed position within an eye in an absence of accommodative reflex; and
wherein the anterior intraocular lens is configured to at least one of change shape or change position during accommodative reflex in order to increase a dioptric power of the accommodating intraocular lens system.

2. The accommodating intraocular lens system of claim 1, wherein:
the posterior intraocular lens is more rigid than the anterior intraocular lens; and
the anterior intraocular lens is more flexible or elastic than the posterior intraocular lens.

3. The accommodating intraocular lens system of claim 2, wherein:
a portion of the anterior intraocular lens is configured to form a larger gap between the anterior intraocular lens and the posterior intraocular lens during accommodative reflex; and
the portion of the anterior intraocular lens is configured to form a smaller gap or no gap between the anterior intraocular lens and the posterior intraocular lens otherwise.

4. The accommodating intraocular lens system of claim 3, wherein the anterior intraocular lens is configured to bulge outward during accommodative reflex to form the larger gap.

5. The accommodating intraocular lens system of claim 1, wherein:
the posterior intraocular lens comprises acrylic; and
the anterior intraocular lens comprises silicone.

6. The accommodating intraocular lens system of claim 1, wherein the second haptics comprise:
projections extending from the second optical lens; and
ridges configured to contact at least one edge of the posterior intraocular lens.

7. The accommodating intraocular lens system of claim 1, wherein the first optical lens represents a plano optical lens.

8. The accommodating intraocular lens system of claim 1, wherein the first and second optical lenses collectively provide a desired amount of dioptric power of the accommodating intraocular lens system.

9. The accommodating intraocular lens system of claim 1, wherein the change in the shape and/or the change in the position of the anterior intraocular lens is defined by (i) an increase in separation of the second optical lens from the first optical lens and (ii) an increase in bending of the second haptics backwards during accommodative reflex.

10. An accommodating intraocular lens system comprising:
a posterior intraocular lens comprising a first optical lens and first haptics; and
an anterior intraocular lens comprising a second optical lens and second haptics, the second haptics attached to the posterior intraocular lens;
wherein the posterior intraocular lens is configured to be moved forward and backward in an eye based on a presence or absence of accommodative reflex;
wherein the posterior intraocular lens is more rigid than the anterior intraocular lens and the anterior intraocular lens is more flexible or elastic than the posterior intraocular lens; and
wherein the anterior intraocular lens is configured to at least one of change shape or change position during accommodative reflex in order to increase a dioptric power of the accommodating intraocular lens system.

11. The accommodating intraocular lens system of claim 10, wherein:
a portion of the anterior intraocular lens is configured to form a larger gap between the anterior intraocular lens and the posterior intraocular lens during accommodative reflex; and
the portion of the anterior intraocular lens is configured to form a smaller gap or no gap between the anterior intraocular lens and the posterior intraocular lens otherwise.

12. The accommodating intraocular lens system of claim 11, wherein the anterior intraocular lens is configured to bulge outward to form the larger gap.

13. The accommodating intraocular lens system of claim 10, wherein:
the posterior intraocular lens comprises acrylic; and
the anterior intraocular lens comprises silicone.

14. The accommodating intraocular lens system of claim 10, wherein the second haptics comprise:
projections extending from the second optical lens; and
ridges configured to contact at least one edge of the posterior intraocular lens.

15. The accommodating intraocular lens system of claim 10, wherein the first optical lens represents a plano optical lens.

16. The accommodating intraocular lens system of claim 10, wherein the first and second optical lenses collectively provide a desired amount of dioptric power of the accommodating intraocular lens system.

17. The accommodating intraocular lens system of claim 10, wherein the posterior intraocular lens is configured to remain in a substantially fixed position within an eye in an absence of accommodative reflex.

18. An accommodating intraocular lens system comprising:
a posterior intraocular lens comprising a first optical lens and first haptics; and
an anterior intraocular lens comprising a second optical lens and second haptics, the second haptics attached to the posterior intraocular lens;
wherein the posterior intraocular lens is configured to be moved forward and backward in an eye based on a presence or absence of accommodative reflex;
wherein the posterior intraocular lens is more rigid than the anterior intraocular lens and the anterior intraocular lens is more flexible or elastic than the posterior intraocular lens;
wherein a portion of the anterior intraocular lens is configured to form a larger gap between the anterior intraocular lens and the posterior intraocular lens during accommodative reflex;
wherein the portion of the anterior intraocular lens is configured to form a smaller gap or no gap between the anterior intraocular lens and the posterior intraocular lens otherwise; and
wherein the anterior intraocular lens is configured to move or bulge outward to form the larger gap.

19. The accommodating intraocular lens system of claim 18, wherein:
the posterior intraocular lens comprises acrylic; and
the anterior intraocular lens comprises silicone.

20. The accommodating intraocular lens system of claim 18, wherein the second haptics comprise:
projections extending from the second optical lens; and
ridges configured to contact at least one edge of the posterior intraocular lens.
